SEQUENCE LISTING (105 sequences)

SEQ ID NO: 1 - DNA, 4978 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 2 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 3 - protein, 9 aa, Artificial
  An artificially synthesized peptide sequence
RYLEQLHQL

SEQ ID NO: 4 - protein, 9 aa, Artificial
  An artificially synthesized peptide sequence
RYLEKPMEI

SEQ ID NO: 5 - protein, 9 aa, Artificial
  An artificially synthesized peptide sequence
KFPELNYQL

SEQ ID NO: 6 - protein, 9 aa, Artificial
  An artificially synthesized peptide sequence
LYQHNLRRI

SEQ ID NO: 7 - protein, 9 aa, Artificial
  An artificially synthesized peptide sequence
RFLQESNVL

SEQ ID NO: 8 - protein, 9 aa, Artificial
  An artificially synthesized peptide sequence
RIVELFRNL

SEQ ID NO: 9 - protein, 9 aa, Artificial
  An artificially synthesized peptide sequence
QFTTKVRLL

SEQ ID NO: 10 - protein, 9 aa, Artificial
  An artificially synthesized peptide sequence
DFDFNYKTL

SEQ ID NO: 11 - protein, 9 aa, Artificial
  An artificially synthesized peptide sequence
KQQMLEQHL

SEQ ID NO: 12 - protein, 9 aa, Artificial
  An artificially synthesized peptide sequence
KMQQLEQML

SEQ ID NO: 13 - protein, 9 aa, Artificial
  An artificially synthesized peptide sequence
KIMDATNIL

SEQ ID NO: 14 - protein, 9 aa, Artificial
  An artificially synthesized peptide sequence
RLLVKFPEL

SEQ ID NO: 15 - protein, 9 aa, Artificial
  An artificially synthesized peptide sequence
SFPMELRQF

SEQ ID NO: 16 - protein, 9 aa, Artificial
  An artificially synthesized peptide sequence
KSQGDMQDL

SEQ ID NO: 17 - protein, 9 aa, Artificial
  An artificially synthesized peptide sequence
RLENWITSL

SEQ ID NO: 18 - protein, 9 aa, Artificial
  An artificially synthesized peptide sequence
RCLWEESRL

SEQ ID NO: 19 - protein, 9 aa, Artificial
  An artificially synthesized peptide sequence
RGSRKFNIL

SEQ ID NO: 20 - protein, 9 aa, Artificial
  An artificially synthesized peptide sequence
FPMELRQFL

SEQ ID NO: 21 - protein, 10 aa, Artificial
  An artificially synthesized peptide sequence
LYSDSFPMEL

SEQ ID NO: 22 - protein, 10 aa, Artificial
  An artificially synthesized peptide sequence
VYHQGLKIDL

SEQ ID NO: 23 - protein, 10 aa, Artificial
  An artificially synthesized peptide sequence
NYQLKIKVCI

SEQ ID NO: 24 - protein, 10 aa, Artificial
  An artificially synthesized peptide sequence
GYKIMDATNI

SEQ ID NO: 25 - protein, 10 aa, Artificial
  An artificially synthesized peptide sequence
SFPMELRQFL

SEQ ID NO: 26 - protein, 10 aa, Artificial
  An artificially synthesized peptide sequence
RYLEQLHQLY

SEQ ID NO: 27 - protein, 10 aa, Artificial
  An artificially synthesized peptide sequence
QFSSTTKRGL

SEQ ID NO: 28 - protein, 10 aa, Artificial
  An artificially synthesized peptide sequence
RYLEKPMEIA

SEQ ID NO: 29 - protein, 10 aa, Artificial
  An artificially synthesized peptide sequence
RQQIKKLEEL

SEQ ID NO: 30 - protein, 10 aa, Artificial
  An artificially synthesized peptide sequence
RSIVSELAGL

SEQ ID NO: 31 - protein, 10 aa, Artificial
  An artificially synthesized peptide sequence
RCLWEESRLL

SEQ ID NO: 32 - protein, 10 aa, Artificial
  An artificially synthesized peptide sequence
HNLRRIKQFL

SEQ ID NO: 33 - protein, 10 aa, Artificial
  An artificially synthesized peptide sequence
SAMEYVQKTL

SEQ ID NO: 34 - protein, 10 aa, Artificial
  An artificially synthesized peptide sequence
LFRNLMKSAF

SEQ ID NO: 35 - protein, 9 aa, Artificial
  An artificially synthesized peptide sequence
GLLSAMEYV

SEQ ID NO: 36 - protein, 9 aa, Artificial
  An artificially synthesized peptide sequence
NLMKSAFVV

SEQ ID NO: 37 - protein, 9 aa, Artificial
  An artificially synthesized peptide sequence
ILVSPLVYL

SEQ ID NO: 38 - protein, 9 aa, Artificial
  An artificially synthesized peptide sequence
RLLVKFPEL

SEQ ID NO: 39 - protein, 9 aa, Artificial
  An artificially synthesized peptide sequence
CLWEESRLL

SEQ ID NO: 40 - protein, 9 aa, Artificial
  An artificially synthesized peptide sequence
WLDNIIDLV

SEQ ID NO: 41 - protein, 9 aa, Artificial
  An artificially synthesized peptide sequence
MLTNNPKNV

SEQ ID NO: 42 - protein, 9 aa, Artificial
  An artificially synthesized peptide sequence
QLYSDSFPM

SEQ ID NO: 43 - protein, 9 aa, Artificial
  An artificially synthesized peptide sequence
GTWDQVAEV

SEQ ID NO: 44 - protein, 9 aa, Artificial
  An artificially synthesized peptide sequence
KGFSFWVWL

SEQ ID NO: 45 - protein, 9 aa, Artificial
  An artificially synthesized peptide sequence
KIMDATNIL

SEQ ID NO: 46 - protein, 9 aa, Artificial
  An artificially synthesized peptide sequence
KLEELQQKV

SEQ ID NO: 47 - protein, 9 aa, Artificial
  An artificially synthesized peptide sequence
KMQQLEQML

SEQ ID NO: 48 - protein, 9 aa, Artificial
  An artificially synthesized peptide sequence
FPMELRQFL

SEQ ID NO: 49 - protein, 9 aa, Artificial
  An artificially synthesized peptide sequence
AQWNQLQQL

SEQ ID NO: 50 - protein, 9 aa, Artificial
  An artificially synthesized peptide sequence
SLSAEFKHL

SEQ ID NO: 51 - protein, 9 aa, Artificial
  An artificially synthesized peptide sequence
NILGTNTKV

SEQ ID NO: 52 - protein, 9 aa, Artificial
  An artificially synthesized peptide sequence
IMDATNILV

SEQ ID NO: 53 - protein, 9 aa, Artificial
  An artificially synthesized peptide sequence
KQFLQSRYL

SEQ ID NO: 54 - protein, 9 aa, Artificial
  An artificially synthesized peptide sequence
SLAESQLQT

SEQ ID NO: 55 - protein, 9 aa, Artificial
  An artificially synthesized peptide sequence
VLSWQFSST

SEQ ID NO: 56 - protein, 9 aa, Artificial
  An artificially synthesized peptide sequence
QQLNNMSFA

SEQ ID NO: 57 - protein, 9 aa, Artificial
  An artificially synthesized peptide sequence
CLDRLENWI

SEQ ID NO: 58 - protein, 9 aa, Artificial
  An artificially synthesized peptide sequence
MLEERIVEL

SEQ ID NO: 59 - protein, 9 aa, Artificial
  An artificially synthesized peptide sequence
YLKTKFICV

SEQ ID NO: 60 - protein, 9 aa, Artificial
  An artificially synthesized peptide sequence
RLLQTAATA

SEQ ID NO: 61 - protein, 9 aa, Artificial
  An artificially synthesized peptide sequence
YQLKIKVCI

SEQ ID NO: 62 - protein, 9 aa, Artificial
  An artificially synthesized peptide sequence
QQLEQMLTA

SEQ ID NO: 63 - protein, 9 aa, Artificial
  An artificially synthesized peptide sequence
MLEQHLQDV

SEQ ID NO: 64 - protein, 9 aa, Artificial
  An artificially synthesized peptide sequence
ALWNEGYIM

SEQ ID NO: 65 - protein, 9 aa, Artificial
  An artificially synthesized peptide sequence
QMLTALDQM

SEQ ID NO: 66 - protein, 9 aa, Artificial
  An artificially synthesized peptide sequence
IVTEELHLI

SEQ ID NO: 67 - protein, 9 aa, Artificial
  An artificially synthesized peptide sequence
IMGYKIMDA

SEQ ID NO: 68 - protein, 9 aa, Artificial
  An artificially synthesized peptide sequence
VQFTTKVRL

SEQ ID NO: 69 - protein, 9 aa, Artificial
  An artificially synthesized peptide sequence
VVTEKQQML

SEQ ID NO: 70 - protein, 9 aa, Artificial
  An artificially synthesized peptide sequence
QMPNAWASI

SEQ ID NO: 71 - protein, 9 aa, Artificial
  An artificially synthesized peptide sequence
ILSTKPPGT

SEQ ID NO: 72 - protein, 9 aa, Artificial
  An artificially synthesized peptide sequence
QLTTLAEKL

SEQ ID NO: 73 - protein, 10 aa, Artificial
  An artificially synthesized peptide sequence
QMLEQHLQDV

SEQ ID NO: 74 - protein, 10 aa, Artificial
  An artificially synthesized peptide sequence
KIMDATNILV

SEQ ID NO: 75 - protein, 10 aa, Artificial
  An artificially synthesized peptide sequence
MAGKGFSFWV

SEQ ID NO: 76 - protein, 10 aa, Artificial
  An artificially synthesized peptide sequence
NMLTNNPKNV

SEQ ID NO: 77 - protein, 10 aa, Artificial
  An artificially synthesized peptide sequence
WVWLDNIIDL

SEQ ID NO: 78 - protein, 10 aa, Artificial
  An artificially synthesized peptide sequence
NILVSPLVYL

SEQ ID NO: 79 - protein, 10 aa, Artificial
  An artificially synthesized peptide sequence
QQLEQMLTAL

SEQ ID NO: 80 - protein, 10 aa, Artificial
  An artificially synthesized peptide sequence
KEGGVTFTWV

SEQ ID NO: 81 - protein, 10 aa, Artificial
  An artificially synthesized peptide sequence
GLSIEQLTTL

SEQ ID NO: 82 - protein, 10 aa, Artificial
  An artificially synthesized peptide sequence
VLSWQFSSTT

SEQ ID NO: 83 - protein, 10 aa, Artificial
  An artificially synthesized peptide sequence
GQFESLTFDM

SEQ ID NO: 84 - protein, 10 aa, Artificial
  An artificially synthesized peptide sequence
NIIDLVKKYI

SEQ ID NO: 85 - protein, 10 aa, Artificial
  An artificially synthesized peptide sequence
SLSAEFKHLT

SEQ ID NO: 86 - protein, 10 aa, Artificial
  An artificially synthesized peptide sequence
VLYQHNLRRI

SEQ ID NO: 87 - protein, 10 aa, Artificial
  An artificially synthesized peptide sequence
SLPVVVISNI

SEQ ID NO: 88 - protein, 10 aa, Artificial
  An artificially synthesized peptide sequence
QLQQLDTRYL

SEQ ID NO: 89 - protein, 10 aa, Artificial
  An artificially synthesized peptide sequence
KQQLNNMSFA

SEQ ID NO: 90 - protein, 10 aa, Artificial
  An artificially synthesized peptide sequence
QLTTLAEKLL

SEQ ID NO: 91 - protein, 10 aa, Artificial
  An artificially synthesized peptide sequence
SLIVTEELHL

SEQ ID NO: 92 - protein, 10 aa, Artificial
  An artificially synthesized peptide sequence
QLNNMSFAEI

SEQ ID NO: 93 - protein, 10 aa, Artificial
  An artificially synthesized peptide sequence
KMQQLEQMLT

SEQ ID NO: 94 - protein, 10 aa, Artificial
  An artificially synthesized peptide sequence
RLLQTAATAA

SEQ ID NO: 95 - protein, 10 aa, Artificial
  An artificially synthesized peptide sequence
LVIKTGVQFT

SEQ ID NO: 96 - protein, 10 aa, Artificial
  An artificially synthesized peptide sequence
WITSLAESQL

SEQ ID NO: 97 - protein, 10 aa, Artificial
  An artificially synthesized peptide sequence
FPMELRQFLA

SEQ ID NO: 98 - protein, 10 aa, Artificial
  An artificially synthesized peptide sequence
KTGVQFTTKV

SEQ ID NO: 99 - protein, 10 aa, Artificial
  An artificially synthesized peptide sequence
ILALWNEGYI

SEQ ID NO: 100 - protein, 10 aa, Artificial
  An artificially synthesized peptide sequence
KLLGPGVNYS

SEQ ID NO: 101 - protein, 10 aa, Artificial
  An artificially synthesized peptide sequence
PMLEERIVEL

SEQ ID NO: 102 - protein, 10 aa, Artificial
  An artificially synthesized peptide sequence
ALDQMRRSIV

SEQ ID NO: 103 - protein, 10 aa, Artificial
  An artificially synthesized peptide sequence
RIVELFRNLM

SEQ ID NO: 104 - protein, 10 aa, Artificial
  An artificially synthesized peptide sequence
AGLLSAMEYV

SEQ ID NO: 105 - protein, 10 aa, Artificial
  An artificially synthesized peptide sequence
IMGYKIMDAT

(19) United States
(12) Patent Application Publication
(10) Pub. No.: US 2011/0052614 A1
(43) Pub. Date: Mar. 3, 2011

(54) STAT3 EPITOPE PEPTIDES

(75) Inventors: Takuya Tsunoda, Kanagawa (JP); Ryuji Osawa, Kanagawa (JP)

(73) Assignee: Oncotherapy Science Inc., Kanagawa (JP)

(21) Appl. No.: 12/745,234

(22) PCT Filed: Nov. 27, 2008
(86) PCT No.: PCT/JP2008/003497

§ 371 Date: Oct. 29, 2010

Related U.S. Application Data
(60) Provisional application No. 60/990,877, filed on Nov. 28, 2007.

(51) Int. Cl. A61K 39/00; C07K 7/06; A61K 38/08; C12N 5/078; C12N 5/0783; A61P 35/00; A61P 37/04

(52) U.S. Cl. 424/185.1; 530/328; 514/19.3; 435/325; 435/377

(57) ABSTRACT

The present invention provides peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, and peptides comprising one of the above-mentioned amino acid sequences with substitution or addition of one, two, or several amino acids, and having cytotoxic T cell inducibility, and also provides drugs comprising these peptides. The peptides of this invention can be used as vaccines.

### CROSS REFERENCE TO RELATED APPLICATIONS

The present application is related to U.S. Ser. No. 60/990,877, filed Nov. 28, 2007 which is incorporated herein by reference.

### TECHNICAL FIELD

1. Field of the Invention

The present invention relates to the field of biological science, more specifically to the field of cancer therapy. In particular, the present invention relates to novel peptides that serve as extremely effective cancer vaccines, and drugs containing such peptides for treating and preventing tumors.

2. Background Art

It has been demonstrated that CD8⁺ cytotoxic T lymphocytes (CTLs) recognize epitope peptides derived from tumor-associated antigens (TAAs) on MHC class I molecule, and kill tumor cells. Since the discovery of the MAGE family as the first example of TAAs, many other TAAs have been discovered using immunological approaches (Boon T. Int J Cancer 54: 177-180, 1993; Boon T, and van der Bruggen P. J Exp Med 183: 725-729, 1996; van der Bruggen P, et al. Science 254: 1643-1647, 1991; Brichard V, et al. J Exp Med 178: 489-495, 1993; Kawakami Y, et al. J Exp Med 180: 347-352, 1994), and some of them are now in the process of clinical development as targets of immunotherapy.

However, so far the clinical efficacy has been low as measured by obvious tumor regression (Rosenberg S A, et al. Nature Med. 10:909-915, 2004). One of the major reasons is a poor immune response of tumor-infiltrating lymphocytes (TIL) and peripheral blood lymphocytes (PBL) in patients with advanced-stage cancer (Miescher S, et al. J Immunol 136:1899-1907, 1986). This tumor-induced immunosuppression is the reason for poor response to tumor antigens (Young R C, et al. Am J Med 52:63-68, 1972), poor proliferation of T cells (Alexander J P, et al. Cancer Res 53:1380-1387, 1997), loss of cytokine production (Horiguchi S, et al. Cancer Res. 59:2950-2956, 1999), and defective signal transduction of T cells and natural killer cells (Kono K, et al. Clin Cancer Res. 11:1825-1828, 1996; Kiessling R, et al. Cancer Immunol Immunother. 48:353-362, 1999). In tumor immunotherapy, the control of immunosuppression in a tumor microenvironment is the most important problem.

STAT3, a member of the STAT family transcription factors, regulates a number of crucial pathways in tumorigenesis including cell cycle progression, invasion and metastasis, tumor angiogenesis and tumor cell evasion of the immune system (Dauer D J, et al. Oncogene. 24: 3397-3408, 2005; Niu G, et al. Oncogene. 21:2000-2008, 2002; Huang S. Clin Cancer Res. 13:1362-1366, 2007). Recently, it has been reported that immature myeloid cells and regulatory T cells, which have a functional activity to suppress anti-tumor immune response, up-regulate STAT3 activity (Yu H, et al. Nat Rev Immunol. 7: 41-51, 2007; Kortylewski M, et al. Nature Med. 11:1314-1321, 2005; Jing N and Tweardy D J. Anti-Cancer Drugs. 16: 601-607, 2005).

### DISCLOSURE OF INVENTION

### Summary of the Invention

The present invention is based, at least in part, on the identification of specific epitope peptides derived from the gene product of STAT3 that possess the ability to elicit cytotoxic T lymphocytes (CTLs) specific to the respective gene product. Peripheral Blood Mononuclear Cells (PBMC) of a healthy donor were stimulated with HLA-A*24 and HLA-A*02 binding peptides derived from STAT3. These peptides are HLA-A24 or HLA-A2 restricted epitope peptides that have the ability to induce potent and specific immune responses against STAT3-expressing immature myeloid cells and regulatory T cells.

Accordingly, the present invention provides methods for regulating (e.g., inhibiting) immunosuppression, which comprise the step of administering STAT3 polypeptides of the invention. Immunosuppression is regulated by the administration of STAT3 polypeptides. Thus, the present invention provides methods for regulating immunosuppression, which comprise the step of administering STAT3 polypeptides. The invention further provides pharmaceutical compositions comprising the STAT3 polypeptides for regulating immunosuppression.

It is to be understood that both the foregoing summary of the invention and the following detailed description are of preferred embodiments, and not restrictive of the invention or other alternate embodiments of the invention.

### BRIEF DESCRIPTION OF THE DRAWINGS

FIG. 1A depicts the result of an IFN-gamma ELISPOT assay for the screening of epitope peptides, which demonstrates that STAT3-A24-9-13, -9-93, -9-354, -9-78, -9-70, -9-308, -9-344, -9-171, -9-140, and -9-658, are potent producers of IFN-gamma. In comparison with the control, cells in the following wells showed potent IFN-gamma production: No. 2 and No. 8 stimulated with STAT3-A24-9-13, No. 5 and No. 9 stimulated with STAT3-A24-9-93, No. 5 stimulated with STAT3-A24-9-354, No. 4 and No. 5 stimulated with STAT3-A24-9-78, No. 3 stimulated with STAT3-A24-9-70, No. 7 stimulated with STAT3-A24-9-308, No. 1 stimulated with STAT3-A24-9-344, No. 6 stimulated with STAT3-A24-9-171, No. 2 stimulated with STAT3-A24-9-140, and No. 1 stimulated with STAT3-A24-9-658, which are indicated by boxes.

FIG. 1B depicts the result of an IFN-gamma ELISPOT assay for the screening of epitope peptides, which demonstrates that STAT3-A24-9-350, -9-180, -9-262, -9-379, -9-26, -10-21, -10-445, and -10-13, are potent producers of IFN-gamma. In comparison with the control, cells in the following wells showed potent IFN-gamma production: No. 7 stimulated with STAT3-A24-9-350, No. 6 and No. 13 stimulated with STAT3-A24-9-180, No. 1, No. 6 and No. 12 stimulated with STAT3-A24-9-262, No. 8 stimulated with STAT3-A24-9-379, No. 8 stimulated with STAT3-A24-9-26, No. 2 stimulated with STAT3-A24-10-21, No. 2 stimulated with STAT3-A24-10-445, and No. 3 stimulated with STAT3-A24-10-13, which are indicated by boxes.

FIG. 1C depicts the result of an IFN-gamma ELISPOT assay for the screening of epitope peptides, which demonstrates that STAT3-A24-10-511, -10-278 and -10-215 are potent producers of IFN-gamma. In comparison with the control, cells in the following wells showed potent IFN-gamma production: No. 1 stimulated with STAT3-A24-10-511, No. 4 stimulated with STAT3-A24-10-278 and No. 1 stimulated with STAT3-A24-10-215, which are indicated by boxes.

FIG. 2A depicts the result of an IFN-gamma ELISPOT assay for the screening of epitope peptides, which demonstrates that STAT3-A2-9-705, -9-360, -9-143, -9-578, -9-205, -9-431, -9-654, -9-343, -9-136, and -9-469 are potent producers of IFN-gamma. In comparison with the control, cells in the following wells showed potent IFN-gamma production: No. 4 and No. 5 stimulated with STAT3-A2-9-705, No. 2, No. 3, No. 4, No. 5 and No. 6 stimulated with STAT3-A2-9-360, No. 1 and No. 6 stimulated with STAT3-A2-9-143, No. 5, No. 6 and No. 8 stimulated with STAT3-A2-9-578, No. 1 and No. 4 stimulated with STAT3-A2-9-205, No. 6 and No. 8 stimulated with STAT3-A2-9-431, No. 7 stimulated with STAT3-A2-9-654, No. 4, No. 5 and No. 7 stimulated with STAT3-A2-9-343, No. 6 stimulated with STAT3-A2-9-136, and No. 6, No. 7 and No. 8 stimulated with STAT3-A2-9-469, which are indicated by boxes.

FIG. 2B depicts the result of an IFN-gamma ELISPOT assay for the screening of epitope peptides, which demonstrates that STAT3-A2-9-524, -10-142, -10-658, -10-554, -10-562, -10-750, -10-114, -10-266, -10-26, -10-340 and -10-308 are potent producers of IFN-gamma. In comparison with the control, cells in the following wells showed potent IFN-gamma production: No. 4 stimulated with STAT3-A2-9-524, No. 7 stimulated with STAT3-A2-10-142, No. 4, No. 6, No. 7 and No. 8 stimulated with STAT3-A2-10-658, No. 3, No. 5 and No. 6 stimulated with STAT3-A2-10-554, No. 7 stimulated with STAT3-A2-10-562, No. 4 stimulated with STAT3-A2-10-750, No. 2 stimulated with STAT3-A2-10-114, No. 8 stimulated with STAT3-A2-10-266, No. 1 and No. 6 stimulated with STAT3-A2-10-26, No. 2, No. 5, No. 6 and No. 8 stimulated with STAT3-A2-10-340 and No. 7 and No. 8 stimulated with STAT3-A2-10-308, which are indicated by boxes.

FIG. 3 depicts the establishment of CTL lines stimulated with STAT3-A24-9-93, STAT3-A24-9-350, STAT3-A24-9-180, STAT3-A24-9-262, STAT3-A24-9-26, STAT3-A24-10-21, STAT3-A2-9-343 and STAT3-A2-10-114. The following CTL lines showed potent IFN-gamma production ability: No. 5 (stimulation of STAT3-A24-9-93), No. 7 (stimulation of STAT3-A24-9-350), No. 6 (stimulation of STAT3-A24-9-180), No. 1 (stimulation of STAT3-A24-9-262), No. 8 (stimulation of STAT3-A24-9-26), No. 2 (stimulation of STAT3-A24-10-21), No. 4 (stimulation of STAT3-A2-9-343) and No. 2 (stimulation of STAT3-A2-10-114). The quantity of IFN-gamma correlated with the ratio of CTL line which refers CTL (responder:R)-peptide pulsed cells (Stimulator:S) ratio (R/S), while IFN-gamma was hardly detected in the control. In the figure, “+” indicates that the cells in the wells were pulsed with the appropriate peptide, and “−” indicates that the cells were not pulsed with a peptide.

FIG. 4 depicts the establishment of CTL clone stimulated with STAT3-A24-10-21. The CTL clone of No. 2-174 (stimulation of STAT3-A24-10-21) showed potent IFN-gamma production ability. The quantity of IFN-gamma correlated with the ratio of CTL line which refers CTL (responder:R)—peptide pulsed cells (Stimulator:S) ratio (R/S), while IFN-gamma was hardly detected in the control. In the figure, “+” indicates that the cells in the wells were pulsed with the appropriate peptide, and “−” indicates that the cells were not pulsed with a peptide.

### DETAILED DESCRIPTION OF THE INVENTION

The words “a”, “an”, and “the” as used herein mean “at least one” unless otherwise specifically indicated.

The terms “cytotoxic T cell” and “cytotoxic T lymphocyte (CTL)” are used inter-changeably herein to refer to a T lymphocyte having the interferon-gamma (IFN-gamma) production ability or the cytolytic ability.

Peptide Derived from STAT3 Having Cytotoxic T Cell Inducibility

Identification of new TAAs that induce potent and specific anti-tumor immune responses warrants further development of clinical applications of peptide vaccination strategy for various types of cancer (Boon T et al., J Exp Med 183: 725-729, 1996; van der Bruggen P et al., Science 254: 1643-1647, 1991; Brichard V et al., J Exp Med 178: 489-495, 1993; Kawakami Y et al., J Exp Med 180: 347-352, 1994; Shichijo S et al., J Exp Med 187:277-288, 1998; Chen Y. T. et al. Proc. Natl. Acd. Sci. USA, 94: 1914-1918, 1997; Harris C C, J Natl Cancer Inst 88:1442-1445, 1996; Butterfield L H et al, Cancer Res 59:3134-3142, 1999; Vissers J L M et al, Cancer Res 59: 5554-5559, 1999; Van der Burg S H et al, J. Immunol. 156:3308-3314, 1996; Tanaka F et al, Cancer Res 57:4465-4468, 1997; Fujie T et al. Int J Cancer 80:169-172, 1999; Kikuchi M et al, Int J Cancer 81: 459-466, 1999; Oiso M et al, Int J Cancer 81:387-394, 1999).

As noted above, various types of antigen specific immunotherapy have been performed; however, clinical efficacy has not been as high as hoped. (Rosenberg S A et al. Nat. Med. 10:909-915, 2004). To improve the clinical efficacy for immunotherapy, it is important to overcome the immunosuppressive factors induced by tumor. It has been reported that tumor-infiltrating immature myeloid cells, immature dendritic cells and immunosuppressive cells against anti-tumor immune systems, have high expression of STAT3 (Yu H, et al. Nat Rev Immunol. 7: 41-51, 2007; Kortylewski M, et al. Nature Med. 11:1314-1321, 2005; Jing N and Tweardy D J. Anti-Cancer Drugs. 16: 601-607, 2005). Recent studies have shown that blocking STAT3 signaling reduced the number of immature DCs and accelerated DC functional maturation (Wang T, et al. Nature Med. 10: 48-54, 2004). Thus, the strategy that involves controlling STAT3 expressing cells is a high potential tool for cancer immunotherapy.

In the present invention, peptides derived from STAT3 were shown to be antigen epitopes restricted by HLA-A24 or HLA-A2, which are common HLA alleles in the human population (Date Y et al. Tissue Antigens, 47: 93-101, 1996, Kondo A et al. J Immunol, 155: 4307-4312, 1995, Kubo R T. et al. J Immunol, 152: 3913-3924, 1994). Candidates of HLA-A24 and HLA-A2 binding peptides derived from STAT3 were identified using information on their binding affinities to HLA-A24 and HLA-A2. After the in vitro stimulation of T-cells by dendritic cells (DCs) loaded with these peptides, CTLs were successfully established using

STAT3-A24-9-13 (SEQ ID NO: 3),

STAT3-A24-9-93 (SEQ ID NO: 4),

STAT3-A24-9-354 (SEQ ID NO: 5),

STAT3-A24-9-78 (SEQ ID NO: 6),

STAT3-A24-9-70 (SEQ ID NO: 7),

STAT3-A24-9-308 (SEQ ID NO: 8),

STAT3-A24-9-344 (SEQ ID NO: 9),

STAT3-A24-9-171 (SEQ ID NO: 10),

STAT3-A24-9-140 (SEQ ID NO: 11),

STAT3-A24-9-658 (SEQ ID NO: 13),

STAT3-A24-9-350 (SEQ ID NO: 14),

STAT3-A24-9-180 (SEQ ID NO: 16),

STAT3-A24-9-262 (SEQ ID NO: 17),

STAT3-A24-9-379 (SEQ ID NO: 19),

STAT3-A24-9-26 (SEQ ID NO: 20),

STAT3-A24-10-21 (SEQ ID NO: 21),

STAT3-A24-10-445 (SEQ ID NO: 22),

STAT3-A24-10-13 (SEQ ID NO: 26),

STAT3-A24-10-511 (SEQ ID NO: 27),

STAT3-A24-10-278 (SEQ ID NO: 29),

STAT3-A24-10-215 (SEQ ID NO: 30),

STAT3-A2-9-705 (SEQ ID NO: 59),

STAT3-A2-9-360 (SEQ ID NO: 61),

STAT3-A2-9-143 (SEQ ID NO: 63),

STAT3-A2-9-578 (SEQ ID NO: 64),

STAT3-A2-9-205 (SEQ ID NO: 65),

STAT3-A2-9-431 (SEQ ID NO: 66),

STAT3-A2-9-654 (SEQ ID NO: 67),

STAT3-A2-9-343 (SEQ ID NO: 68),

STAT3-A2-9-136 (SEQ ID NO: 69),

STAT3-A2-9-469 (SEQ ID NO: 70),

STAT3-A2-9-524 (SEQ ID NO: 72),

STAT3-A2-10-142 (SEQ ID NO: 73),

STAT3-A2-10-658 (SEQ ID NO: 74),

STAT3-A2-10-554 (SEQ ID NO: 75),

STAT3-A2-10-562 (SEQ ID NO: 77),

STAT3-A2-10-750 (SEQ ID NO: 83),

STAT3-A2-10-114 (SEQ ID NO: 94),

STAT3-A2-10-266 (SEQ ID NO: 96),

STAT3-A2-10-26 (SEQ ID NO: 97),

STAT3-A2-10-340 (SEQ ID NO: 98) and

STAT3-A2-10-308 (SEQ ID NO: 103).

These CTLs showed potent specific CTL activity against the peptide-pulsed target cells. These results strongly suggest that STAT3 is a novel antigen recognized by CTL and that fragment thereof including the above peptides are epitope peptides restricted by HLA-A24 and HLA-A2. Since STAT3 is overexpressed in most cancer patients and associated with immunosuppression and angiogenesis, STAT3 is a good target for immunotherapy to promote immunotherapy efficacy. Thus, the present invention provides a nonapeptide or decapeptide selected from peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103. In preferred embodiments, the present invention provides a peptide having cytotoxic T cell inducibility, wherein the peptide comprises the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, with substitution or addition of one, two, or several amino acids. For example, in the present invention, preferable numbers of amino acid residues to be substituted may be one or two.

Accordingly, the present invention further provides methods of regulating immunosuppression in tumor microenvironment and angiogenesis, said methods comprising steps of administering an immunogenic peptide of less than about 40 amino acids, often less than about 20 amino acids, usually less than about 15 amino acids, comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103. The present invention further provides methods for treating or preventing cancer, said methods comprising steps of administering an immunogenic peptide of less than about 40 amino acids, often less than about 20 amino acids, usually less than about 15 amino acids, comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103. Alternatively, the immunogenic peptide may comprise the sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, in which one, two, or several amino acids are substituted or added. In preferred embodiments, the immunogenic peptide is a nonapeptide or decapeptide.

Alternatively, the present invention provides a method of inducing anti-immunosuppression and anti-angiogenesis, said method comprising steps of administering an immunogenic peptide of the invention comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103. In the present invention, the peptide can be administered to a subject in vivo or ex vivo. Furthermore, the present invention also provides use of a nonapeptide or decapeptide selected from peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, for manufacturing an immunogenic composition for regulating immunosuppression and/or angiogenesis. Alternatively, the present invention also relates to nonapeptides or decapeptides selected from peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, for regulating immunosuppression and/or angiogenesis. In preferred embodiments, use of nonapeptides or decapeptides selected from peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, for manufacturing or preparation of immunological or pharmaceutical composition for treating or preventing a cancer is provided. Alternatively, the immunogenic peptide may comprise the sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, in which one, two, or several amino acids are substituted or added. In preferred embodiments, the immunogenic peptide is a nonapeptide or decapeptide.

Alternatively, the present invention further provides a method or process for manufacturing an immunogenic composition for regulating immunosuppression and/or angiogenesis, wherein the method or process comprises the step of formulating a pharmaceutically or physiologically acceptable carrier with a nonapeptide or decapeptide selected from peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103. Alternatively, the present invention further provides a method or process for manufacturing an immunogenic composition for regulating immunosuppression and/or angiogenesis, wherein the method or process comprises the step of admixing an active ingredient with a pharmaceutically or physiologically acceptable carrier, wherein the active ingredient is a nonapeptide or decapeptide selected from peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103.

Moreover, the present invention further provides a method or process for manufacturing an immunogenic composition for treating a cancer, wherein the method or process comprises the step of formulating a pharmaceutically or physiologically acceptable carrier with a nonapeptide or decapeptide selected from peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103. Alternatively, the present invention further provides a method or process for manufacturing an immunogenic composition for treating or preventing a cancer, wherein the method or process comprises the step of admixing an active ingredient with a pharmaceutically or physiologically acceptable carrier, wherein the active ingredient is a nonapeptide or decapeptide selected from peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103. Alternatively, the immunogenic peptide may comprise the sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, in which one, two, or several amino acids are substituted or added. In preferred embodiments, the immunogenic peptide is a nonapeptide or decapeptide.

Homology analysis of the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103 showed that they do not have a significant homology with peptides derived from any known human gene products. This lowers the possibility of unknown or undesirable immune responses with immunotherapy against these molecules.

Regarding HLA antigens, the use of the A24 and A2 types which are highly expressed among the Japanese and Caucasian is favorable for obtaining effective results, and the use of subtypes such as A2402, A0201 and A0206 is even more preferable. Typically, in the clinic, the type of HLA antigen of the patient requiring treatment is investigated in advance, which enables appropriate selection of peptides that have high levels of binding affinity to the appropriate HLA antigen and CTL inducibility. Furthermore, in order to obtain peptides showing high HLA binding affinity and CTL inducibility, the amino acid sequence of naturally displayed partial STAT3 peptides may be modified by substitution or addition of one, two, or several amino acids. Herein, the term “several” means refers to five or less, more preferably three or less.

Furthermore, in addition to peptides that are naturally displayed, the immunogenic peptides of the invention may be modified based on a known pattern of sequences of peptides displayed upon binding of HLA antigens (J. Immunol., 152, 3913, 1994; Immunogenetics. 41:178, 1995; J. Immunol. 155:4307, 1994). For example, peptides showing high HLA-A24 binding affinity in which the second amino acid from the N terminus is substituted with phenylalanine, tyrosine, methionine, or tryptophan, and/or whose amino acid at the C terminus is substituted with phenylalanine, leucine, isoleucine, tryptophan, or methionine may also be used favorably. Accordingly, the peptides of the present invention selected from group consisting of

STAT3-A24-9-13 (SEQ ID NO: 3),

STAT3-A24-9-93 (SEQ ID NO: 4),

STAT3-A24-9-354 (SEQ ID NO: 5),

STAT3-A24-9-78 (SEQ ID NO: 6),

STAT3-A24-9-70 (SEQ ID NO: 7),

STAT3-A24-9-308 (SEQ ID NO: 8),

STAT3-A24-9-344 (SEQ ID NO: 9),

STAT3-A24-9-171 (SEQ ID NO: 10),

STAT3-A24-9-140 (SEQ ID NO: 11),

STAT3-A24-9-658 (SEQ ID NO: 13),

STAT3-A24-9-350 (SEQ ID NO: 14),

STAT3-A24-9-180 (SEQ ID NO: 16),

STAT3-A24-9-262 (SEQ ID NO: 17),

STAT3-A24-9-379 (SEQ ID NO: 19),

STAT3-A24-9-26 (SEQ ID NO: 20),

STAT3-A24-10-21 (SEQ ID NO: 21),

STAT3-A24-10-445 (SEQ ID NO: 22),

STAT3-A24-10-13 (SEQ ID NO: 26),

STAT3-A24-10-511 (SEQ ID NO: 27),

STAT3-A24-10-278 (SEQ ID NO: 29) and

STAT3-A24-10-215 (SEQ ID NO: 30) may be modified by such manner. Further, peptides obtained from such modification of the amino acid sequence are also used for methods or compositions of the present invention.

On the other hand, peptides in which the second amino acid from the N terminus is substituted with leucine or methionine, and/or in which C-terminal amino acid is substituted with valine or leucine may be preferably used as peptides with high HLA-A0201 binding affinity. For instance, the peptides of the present invention selected from group consisting of

STAT3-A2-9-705 (SEQ ID NO: 59),

STAT3-A2-9-360 (SEQ ID NO: 61),

STAT3-A2-9-143 (SEQ ID NO: 63),

STAT3-A2-9-578 (SEQ ID NO: 64),

STAT3-A2-9-205 (SEQ ID NO: 65),

STAT3-A2-9-431 (SEQ ID NO: 66),

STAT3-A2-9-654 (SEQ ID NO: 67),

STAT3-A2-9-343 (SEQ ID NO: 68),

STAT3-A2-9-136 (SEQ ID NO: 69),

STAT3-A2-9-469 (SEQ ID NO: 70),

STAT3-A2-9-524 (SEQ ID NO: 72),

STAT3-A2-10-142 (SEQ ID NO: 73),

STAT3-A2-10-658 (SEQ ID NO: 74),

STAT3-A2-10-554 (SEQ ID NO: 75),

STAT3-A2-10-562 (SEQ ID NO: 77),

STAT3-A2-10-750 (SEQ ID NO: 83),

STAT3-A2-10-114 (SEQ ID NO: 94),

STAT3-A2-10-266 (SEQ ID NO: 96),

STAT3-A2-10-26 (SEQ ID NO: 97),

STAT3-A2-10-340 (SEQ ID NO: 98) and

STAT3-A2-10-308 (SEQ ID NO: 103) may be modified by such manner. Further, peptides obtained from such modification of the amino acid sequence are also used for methods or compositions of the present invention.

Furthermore, one to two amino acids may also be bound to the N and/or C terminus of the peptides.

Substitutions can be introduced not only at the terminal amino acids but also at the position of potential TCR recognition of peptides. Several studies have demonstrated that amino acid substitutions in a peptide can be equal to or better than the original, for example CAP1, p 53₍₂₆₄₋₂₇₂₎, Her-2/neu₍₃₆₉₋₃₇₇₎ or gp100₍₂₀₉₋₂₁₇₎(Zaremba et al. Cancer Res. 57, 4570-4577, 1997, T. K. Hoffmann et al. J. Immunol. (2002) February 1; 168(3):1338-47, S. O. Dionne et al. Cancer Immunol immunother. (2003) 52: 199-206 and S. O. Dionne et al. Cancer Immunology, Immunotherapy (2004) 53, 307-314).

Such modified peptides with high HLA antigen binding affinity and retained CTL inducibility are also included in the present invention.

However, when the peptide sequence is identical to a portion of the amino acid sequence of an endogenous or exogenous protein having a different function, side effects such as autoimmune disorders or allergic symptoms against specific substances may be induced. Therefore, it is often convenient to avoid situations in which the sequence matches the amino acid sequence of another protein. This can be easily achieved by performing a homology search using available databases. Furthermore, if it is clear from homology searches that not even peptides that differ by one or two amino acids exist, it is unlikely that modifications of the above-mentioned amino acid sequence to increase the binding affinity with HLA antigens, and/or increase the CTL inducibility will impose such problems.

Although peptides having high binding affinity to the HLA antigens as described above are expected to be highly effective, the candidate peptides, which are selected using the presence of high binding affinity as an indicator, must be examined for their actual CTL inducibility. Confirmation of CTL inducibility is accomplished, for example, by inducing antigen-presenting cells carrying human MHC antigens (for example, B-lymphocytes, macrophages, and dendritic cells), or more specifically, dendritic cells derived from human peripheral blood mononuclear leukocytes, stimulating the cells with the peptides, mixing with CD8-positive cells, and then measuring the IFN-gamma produced and released by CTL against the target cells.

As the reaction system, transgenic animals that have been produced to express a human HLA antigen (for example, those described in Hum. Immunol. 2000 August; 61(8):764-79 Induction of CTL response by a minimal epitope vaccine in HLA A*0201/DR1 transgenic mice: dependence on HLA class II restricted T(H) response. BenMohamed L., Krishnan R., Longmate J., Auge C., Low L., Primus J., Diamond D J.) may also be used. For example, the target cells can be radiolabeled with ⁵¹Cr and such, and cytotoxic activity can be calculated from radioactivity released from the target cells. Alternatively, it can be examined by measuring IFN-gamma produced and released by CTL in the presence of antigen-presenting cells that carry immobilized peptides, and visualizing the inhibition zone on the media using anti-IFN-gamma monoclonal antibodies.

The result of examining the CTL inducibility of peptides as described above showed that those having high binding affinity to an HLA antigen showed varying abilities to induce CTL. Furthermore, nonapeptides or decapeptides selected from peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, showed particularly high CTL inducibility.

As noted above, the present invention provides peptides having cytotoxic T cell inducibility, and comprising addition or substitution of one, two, or several amino acids in the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103. It is preferable that amino acid sequences comprising 9 or 10 amino acids indicated in the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, with substitution or addition of one, two, or several amino acids, do not match amino acid sequences of other proteins. In particular, favorable examples are: for HLA-A24, amino acid substitution to phenylalanine, tyrosine, methionine, or tryptophan at the second amino acid from the N terminus, and/or to phenylalanine, leucine, isoleucine, tryptophan, or methionine at the C-terminal amino acid; for HLA-A2, amino acid substitution to leucine or methionine at the second amino acid from the N terminus, and/or to valine or leucine at the C-terminal amino acid, and/or amino acid addition of one or two amino acids at the N terminus and/or C terminus.

Peptides of the invention can be prepared using well known techniques. For example, the peptides can be prepared synthetically by recombinant DNA technology or chemical synthesis. Peptides of the invention may be synthesized individually or as longer polypeptides comprising two or more peptides. The peptide are preferably isolated, i.e., substantially free of other naturally occurring host cell proteins and fragments thereof.

The peptides may contain modifications such as glycosylation, side chain oxidation, or phosphorylation, so long as the modifications do not destroy the biological activity of the peptides as described herein. Other modifications include incorporation of Damino acids or other amino acid mimetics that can be used, for example, to increase the serum half life of the peptides.

Composition for Enhancing the Anti-Cancer Immunity as Vaccine

The peptides of this invention can be prepared in a combination, which comprises two or more of peptides of the invention, for use as a vaccine that may induce CTLs in vivo. The peptides may be in a cocktail or may be conjugated to each other using standard techniques. For example, the peptides can be expressed as a single polypeptide sequence. The peptides in the combination may be the same or different. By administering the peptides of this invention, the peptides are presented at a high density on the HLA antigens of antigen-presenting cells, and then CTLs that specifically react to the complex formed between the displayed peptide and the HLA antigen are induced. Alternatively, antigen presenting cells are obtained by removing dendritic cells from the subject. These cells are then stimulated with the peptides of this invention, and CTLs are induced in the subject by re-administering these cells to the subject. As a result, a response towards the target cells can be enhanced.

More specifically, the present invention provides drugs comprising one or more of peptides of this invention for regulating tumor angiogenesis and immunosuppression by inhibiting regulatory T cells (T-regs). The peptides of this invention can be used for regulating T-regs and angiogenesis.

The term of “regulatory T cells (T-regs)” is a specialized subpopulation of T cells that act as the suppressor of the immunological activity.

The peptides of this invention can be administered directly as a pharmaceutical composition been formulated by conventional formulation methods. In such cases, in addition to the peptides of this invention, carriers, excipients, and such that are commonly used for drugs can be included as appropriate without particular limitations. The immunogenic compositions of this invention may be used for cancer therapy through suppression of angiogenesis and enhancement of cancer immunotherapy by regulating T-regs.

The immunogenic compositions for cancer therapy and enhancement of cancer immunotherapy by suppression of angiogenesis and generation of T-regs, which comprise the peptides of this invention as the active ingredients, can comprise an adjuvant so that cellular immunity will be established effectively, or they may be administered with other active ingredients, and they may be administered by liquid form. Exemplary adjuvants that may be used include those described in the literature (Clin. Microbiol. Rev., 7:277-289, 1994). Such adjuvants include, for example, aluminum phosphate, aluminum hydroxide and alum. Furthermore, liposome formulations, granular formulations in which the drug is bound to few-micrometer diameter beads, and formulations in which a lipid is bound to the peptide may be conveniently used. The method of administration may be oral, intradermal, subcutaneous, intravenous injection, or such, and systemic administration or local administration to the vicinity of the targeted site is possible. The dosage of the peptides of this invention can be adjusted appropriately according to the disease to be treated, age of the patient, weight, method of administration, and such, and is usually 0.001 mg to 1000 mg, preferably 0.001 mg to 1000 mg, more preferably 0.1 mg to 10 mg, and is preferably administered once in a few days to few months. One skilled in the art can appropriately select a suitable dosage.

Alternatively, the present invention provides intracellular vesicles called exosomes, which present on their surface complexes formed between the peptides of this invention and HLA antigens. Exosomes can be prepared, for example, by using the methods described in detail in Published Japanese Translation of International Publication Nos. Hei 11-510507 and 2000-512161, and is preferably prepared using antigen presenting cells obtained from subjects who are targets of treatment and/or prevention. The exosomes of this invention can be used as vaccines, similarly to the peptides of this invention. That is, the present invention provides use of the exosomes of the present invention for manufacturing a pharmaceutical composition for inducing antigen-presenting cells, or for inducing CTLs. Alternatively, the present invention further provides a method or process for manufacturing a pharmaceutical composition comprising the exosome of the present invention for inducing antigen-presenting cells, or for inducing CTLs.

The type of HLA antigens used must match that of the subject requiring treatment and/or prevention. For example, for Japanese, HLA-A24 or HLA-A2, in particular, HLA-A2402 or HLA-A0201 and A0206, respectively, is often appropriate.

In some embodiments, the vaccine compositions of the invention comprise a component which primes cytotoxic T lymphocytes. Lipids have been identified as agents capable of priming CTL in vivo against viral antigens. For example, palmitic acid residues can be attached to the epsilon- and alpha-amino groups of a lysine residue and then linked to an immunogenic peptide of the invention. The lipidated peptide can then be administered either directly in a micelle or particle, incorporated into a liposome, or emulsified in an adjuvant. As another example of lipid priming CTL responses, E. coli lipoproteins, such as tripalmitoyl-S-glycerylcysteinlyseryl-serine (P3CSS), can be used to prime CTL when covalently attached to an appropriate peptide (see, e.g., Deres, et al., Nature 342:561, 1989).

The immunogenic compositions of the invention may also comprise nucleic acids encoding the immunogenic peptides disclosed here (see, e.g., Wolff et al. (1990) Science 247:1465-1468; U.S. Pat. Nos. 5,580,859; 5,589,466; 5,804,566; 5,739,118; 5,736,524; 5,679,647; and WO 98/04720). Examples of DNA-based delivery technologies include “naked DNA”, facilitated (bupivacaine, polymers, peptide-mediated) delivery, cationic lipid complexes, and particle-mediated (“gene gun”), and pressure-mediated delivery (see, e.g., U.S. Pat. No. 5,922,687).

The immunogenic peptides of the invention can also be expressed by viral or bacterial vectors. Examples of expression vectors include attenuated viral hosts, such as vaccinia or fowlpox. This approach involves the use of vaccinia virus, for example, as a vector to express nucleotide sequences that encode the peptides. Upon introduction into a host, the recombinant vaccinia virus expresses the immunogenic peptide, and thereby elicits an immune response. Vaccinia vectors and useful immunization methods are described in, for example, U.S. Pat. No. 4,722,848. Another vector is BCG (Bacille Calmette Guerin). BCG vectors are described in Stover, et al. (1991) Nature 351:456-460. A wide variety of other vectors useful for therapeutic administration or immunization, for example, adeno and adeno-associated virus vectors, retroviral vectors, Salmonella typhi vectors, detoxified anthrax toxin vectors, and the like, will be available (see, e.g., Shata, et al. (2000) Mol. Med. Today 6:66-71; Shedlock, et al. (2000) J. Leukoc. Biol. 68:793-806; and Hipp, et al. (2000) In Vivo 14:571-85).

The present invention also provides methods of inducing antigen-presenting cells using the peptides of this invention. The antigen-presenting cells can be induced by preparing dendritic cells from the peripheral blood monocytes and then contacting (stimulating) them with the peptides of this invention in vitro, ex vivo or in vivo. When the peptides of this invention are administered to a subject, antigen-presenting cells that have the peptides of this invention immobilized to them are induced in the body of the subject. Alternatively, after immobilizing the peptides of this invention to the antigen-presenting cells, the cells can be administered to the subject as a vaccine. For example, the ex vivo administration may comprise the steps of:

a) collecting antigen presenting cells from a subject, and

b) contacting the antigen presenting cells of step a with a peptide.

Alternatively, the present invention provides use of the peptides of this invention for manufacturing a pharmaceutical composition for inducing antigen-presenting cells. Further, the present invention also provides the peptide of the present invention for inducing antigen-presenting cells. Alternatively, the present invention further provides a method or process for manufacturing a pharmaceutical composition comprising the peptide of the present invention for inducing antigen-presenting cells. The antigen presenting cells obtained by step b can be administered to the subject as a vaccine.

This invention also provides a method for inducing antigen-presenting cells having a high level of cytotoxic T cell inducibility, in which the method comprises the step of transferring genes comprising polynucleotides that encode the peptides of this invention into antigen-presenting cells in vitro. The introduced genes may be in the form of DNAs or RNAs. For the method of introduction, without particular limitations, various methods conventionally performed in this field, such as lipofection, electroporation, and the calcium phosphate method, may be used. More specifically, it may be performed as described in Cancer Res., 56:5672-7, 1996; J. Immunol., 161:5607-13, 1998; J. Exp. Med., 184:465-72, 1996; Published Japanese Translation of International Publication No. 2000-509281. By transferring the gene into antigen-presenting cells, the gene undergoes transcription and translation, in the cell, the expressed protein proceeds through a presentation pathway to be presented as partial peptides on the surface of the cell in the context of MHC Class I or Class II molecules.

Furthermore, the present invention provides methods for inducing CTL using the peptides of this invention. When the peptides of this invention are administered to a subject, CTLs are induced in the body of the subject. Therefore, the strength of the immune system is enhanced by targeting the T-regs and angiogenesis around the tumor is suppressed. Alternatively, they may be used for an ex vivo therapeutic method, in which subject-derived antigen-presenting cells, and CD8-positive cells, or peripheral blood mononuclear leukocytes are contacted (stimulated) with the peptides of this invention in vitro, and after CTLs are induced, the cells are returned to the subject. For example, the method may comprise the steps of:

a) collecting antigen presenting cells from a subject,

b) contacting the antigen presenting cells of step a with a peptide,

c) mixing and co-culturing the antigen presenting cells of step b with CD8⁺ T cells to induce cytotoxic T-cells (CTLs), and

d) collecting CD8⁺ T cells from the co-culture of step c.

Alternatively, the present invention provides use of the peptides of this invention for manufacturing a pharmaceutical composition for inducing CTLs. Further, the present invention also provides the peptides of the present invention for inducing CTLs. Alternatively, the present invention further provides a method or process for manufacturing a pharmaceutical composition comprising the polypeptide of the present invention for inducing CTLs. The CTLs having cytotoxic activity obtained by step d can be administered to the subject as a vaccine.

Furthermore, the present invention provides isolated CTLs induced using the peptides of this invention. The CTLs, which have been induced by stimulation of antigen-presenting cells that present the peptides of this invention, are preferably derived from subjects who are targets of treatment and/or prevention; and they can be administered singly, or in combination with other drugs including the peptides of this invention or exosomes for the purpose of regulating induction of CTLs. The obtained CTLs act specifically against target cells that present the peptides of this invention or preferably the same peptides used for induction. The target cells may be cells that express STAT3 endogenously, or cells that are transfected with the STAT3 gene, and cells that present the peptides of this invention on the cell surface due to stimulation by these peptides can also become targets of attack.

The present invention also provides antigen-presenting cells that present complexes formed between HLA antigens and the peptides of this invention. The antigen-presenting cells that are obtained by contacting with the peptides of this invention, or nucleotides encoding the peptides of this invention are preferably derived from subjects who are targets of treatment and/or prevention, and can be administered as vaccines singly or in combination with other drugs including the peptides of this invention, exosomes, or CTLs.

The present invention also provides compositions comprising nucleic acids encoding polypeptides that are capable of forming a subunit of a T cell receptor (TCR), and methods of using the same. The TCR subunits have the ability to form TCRs that confer T cell specificity for STAT3-presenting cells. By using known methods in the art, it is possible to identify the nucleic acids of alpha- and beta-chains as TCR subunits of the CTLs induced with one or more peptides of this invention (WO2007/032255 and Morgan et al., J Immunol, 171, 3288 (2003)). The derivative TCRs preferably bind to target cells displaying the STAT3 peptide with high avidity, and mediate efficient killing of target cells presenting the STAT3 peptide in vivo and in vitro.

Nucleic acids encoding the TCR subunits can be incorporated into suitable vectors, for example, retroviral vectors. These vectors are well known in the art. The nucleic acids or vectors comprising them usefully can be transferred into a T cell, where the T cell is preferably from a patient. Advantageously, the invention provides an off-the-shelf composition that allows rapid modification of a patient's own T cells (or those of another mammal) to rapidly and easily produce modified T cells having excellent cancer cell killing properties.

Also, the present invention provides CTLs which are prepared by transduction with nucleic acids encoding polypeptides of TCR subunits that bind with a STAT3 peptide, for example, SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103, in the context of HLA-A24 or HLA-A2. The transduced CTLs are capable of homing to cancer cells in vivo, and expand by well known culturing method in vitro (e.g., Kawakami et al., J. Immunol., 142, 3452-3461 (1989)). The T cells of the present invention can be used to form an immunogenic composition useful for treating or preventing cancer in a patient in need thereof (WO2006/031221).

In the present invention, the phrase “vaccine” (also referred to as an immunogenic composition) refers to a substance that has the function to inhibit tumor-induced immunosuppression and thereby enhance anti-tumor immunity, when inoculated into animals. A vaccine of the invention may also have the function to induce immunity for T-regs and/or cells relating to angiogenesis. According to the present invention, polypeptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103 can be used to prepare HLA-A24 or HLA-A02 restricted epitope peptides that induce potent and specific immune response against STAT3-expressing T-regs and STAT3-expressing angiogenesis-related cells. Thus, the present invention also encompasses methods of inhibiting tumor-induced immunosuppression and angiogenesis using polypeptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103. In general, inhibiting tumor-induced immunosuppression and angiogenesis include immune responses such as follows:
induction of cytotoxic lymphocytes against STAT3-expressing T-regs and STAT3-expressing angiogenesis-related cells,
induction of antibodies that recognize STAT3-expressing T-regs and the STAT3-expressing angiogenesis-related cells, and
induction of cytokine production to suppressing T-regs and/or angiogenesis-related cells.

Therefore, when a certain protein induces any one of these immune responses upon inoculation into an animal, the protein is determined to inhibit tumor-induced immunosuppression and angiogenesis. The inhibition of tumor-induced immunosuppression and angiogenesis by a protein can be detected by observing the response of the immune system in the host against the protein in vivo or in vitro.

For example, a method for detecting the induction of cytotoxic T lymphocytes is well known. A foreign substance that enters the living body is presented to T cells and B cells by the action of antigen presenting cells (APCs). T cells that respond to the antigen presented by APC in an antigen-specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to a T cell by APC, and detecting the induction of CTLs. Furthermore, APCs have the effect of activating CD4⁺ T cells, CD8⁺ T cells, macrophages, eosinophils and NK cells. Since CD4⁺ T cells are also important in anti-tumor immunity, the anti-tumor immunity inducing action of the peptide can be evaluated using the activation effect of these cells as an indicator.

A method for evaluating the induction of CTLs using dendritic cells (DCs) as APC is well known in the art. DC is a representative APC having the strongest CTL-inducing effect among APCs. In this method, the test polypeptide is initially contacted with DC and then this DC is contacted with T cells. Detection of T cells having cytotoxic effects against the cells of interest after their contact with DC shows that the test polypeptide has an activity of inducing cytotoxic T cells. The activity of CTLs against T-regs and angiogenesis-related cells can be detected, for example, using the lysis of ⁵¹Cr-labeled tumor cells as an indicator. Alternatively, the method of evaluating the degree of T-regs and the damage of angiogenesis-related cells using ³H-thymidine uptake activity or LDH (lactose dehydrogenase) release as an indicator is also well known.

Apart from DC, peripheral blood mononuclear cells (PBMCs) may also be used as an APC. The induction of CTLs is reported to be enhanced by culturing PBMC in the presence of GM-CSF and IL-4. Similarly, CTL has been shown to be induced by culturing PBMC in the presence of keyhole limpet hemocyanin (KLH) and IL-7.

The test polypeptides confirmed to possess CTL-inducing activity by these methods are polypeptides having a DC activating effect and subsequent CTL-inducing activity. Therefore, polypeptides that induce CTLs against T-regs and angiogenesis-related cells are useful as vaccines for cancer therapy and enhancement of cancer immunotherapy. Furthermore, APCs that have acquired the ability to induce CTLs against T-regs and angiogenesis-related cells by contacting with the polypeptides are useful as vaccines for cancer therapy and enhancement of cancer immunotherapy. Furthermore, CTLs that have acquired cytotoxicity due to presentation of polypeptide antigens by APC can be also used as vaccines for cancer therapy and enhancement of cancer immunotherapy. Such regulation methods for T-regs and angiogenesis-related cells using immunity due to APCs and CTLs are referred to as cellular immunotherapy.

Generally, when using a polypeptide for cellular immunotherapy, the efficiency of CTL induction is known to increase by combining a plurality of polypeptides having different structures and contacting them with DC. Therefore, when stimulating DC with protein fragments, it is advantageous to use a mixture of multiple types of fragments.

Alternatively, the inhibition of tumor-induced immunosuppression and angiogenesis by a polypeptide can be confirmed by observing the induction of antibody production against T-regs and angiogenesis-related cells. For example, when antibodies against a polypeptide are induced in a laboratory animal immunized with the polypeptide, and when T-regs and angiogenesis-related cells are suppressed by those antibodies, the polypeptide can be determined to have an ability to inhibit tumor-induced immunosuppression and angiogenesis.

Inhibition of tumor-induced immunosuppression and angiogenesis is induced by administering the vaccine of this invention, and the induction enables dissolution of immunosuppression and angiogenesis. Such effects are preferably statistically significant. For example, when compared to a control without vaccine administration, the regulatory effect of a vaccine against T-regs and angiogenesis-related cells is statistically significant with a significance level of 5% or less. For example, Student's t-test, the Mann-Whitney U-test, or ANOVA may be used for statistical analyses.

The above-mentioned proteins having immunological activity, or a polynucleotide or vector encoding the proteins may be combined with an adjuvant. An adjuvant refers to a compound that enhances the immune response against a protein having immunological activity when administered together (or successively) with the protein. Examples of adjuvants include cholera toxin, salmonella toxin, alum and such, but are not limited thereto. Furthermore, the vaccine of this invention may be combined appropriately with a pharmaceutically acceptable carrier. Examples of such carriers are sterilized water, physiological saline, phosphate buffer, culture fluid and such. Furthermore, the vaccine may contain, as necessary, stabilizers, suspensions, preservatives, surfactants and such. The vaccine is administered systemically or locally. Vaccine administration may be performed in a single administration or boosted by multiple administrations.

When using APCs or CTLs as a vaccine of this invention, T-regs and angiogenesis-related cells can be regulated, for example, by an ex vivo method. More specifically, PBMCs of the subject receiving treatment or prevention are collected and contacted with the polypeptide ex vivo, and following the induction of APCs or CTLs, the cells may be administered to the subject. APCs can be also induced by introducing a vector encoding the polypeptide into PBMCs ex vivo. The APCs or CTLs induced in vitro can be cloned prior to administration. By cloning and growing cells having high activity of damaging target cells, cellular immunotherapy can be performed more effectively. Furthermore, APCs and CTLs isolated in this manner may be used for cellular immunotherapy not only against individuals from whom the cells are derived, but also against similar types of diseases in other individuals.

The following examples are presented to illustrate the present invention and to assist one of ordinary skill in making and using the same. The examples are not intended in any way to otherwise limit the scope of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. Any patents, patent applications, and publications cited herein are incorporated by reference.

### EXAMPLES

The present invention is illustrated in detail by the following Examples, but is not restricted to these Examples.

Materials and Methods

Cell Lines

The A24LCL cell line, human B-lymphoblastoid cell lines, and T2 cell line were purchased from ATCC.

Candidate Selection of Peptide Derived from STAT3

From the amino acid sequence of STAT3 (SEQ ID NO: 2, NP_{—}644805; 770 residues) encoded by the nucleotide sequence of NM_{—}139276 (SEQ ID NO: 1), STAT3-derived 9-mer and 10-mer peptides that bind to the HLA-A*2402 and HLA-A*0201 molecules were predicted by a binding prediction software “BIMAS” (http://bimas.dcrt.nih.gov/cgi-bin/molbio/ken_parker_comboform). These peptides were synthesized by Sigma (Sapporo, Japan) according to the standard solid-phase synthesis method and purified by reverse phase HPLC. The purity (>90%) and identity of the peptides were determined by analytical HPLC and mass spectrometry analysis, respectively. Peptides were dissolved in dimethylsulfoxide (DMSO) at 20 mg/ml and stored at −80 degrees C.

In Vitro CTL Induction

Monocyte-derived dendritic cells (DCs) were used as antigen-presenting cells (APCs) to induce a CTL response against peptides presented on HLA. DCs were generated in vitro as described in Horiguchi S, et al. Cancer Res. 59:2950-2956, 1999. Briefly, peripheral blood mononuclear cells (PBMCs) isolated from a normal subject (HLA-A*2402 and/or HLA-A*0201) in Ficoll-Plaque (Pharmacia) solution were separated by adherence to a plastic tissue culture dish (Becton Dickinson), so as to enrich them for the monocyte fraction. The monocyte-enriched population was cultured in the presence of 1000 U/ml of GM-CSF (R&D System) and 1000 U/ml of IL-4 (R&D System) in AIM-V (Invitrogen) containing 2% heat-inactivated autologous serum (AS). After 7 days in the culture, the cytokine-generated DCs were pulsed with 20 microgram/ml of the synthesized peptides in the presence of 3 microgram/ml of beta 2-microglobulin for 3 hours at 37 degrees C. in AIM-V.

These peptide-pulsed DCs were then inactivated by MMC (30 microgram/ml for 30 mins) and mixed at a 1:20 ratio with autologous CD8⁺ T cells, obtained by positive selection with the CD8 Positive Isolation Kit (Dynal). These cultures were set up in 48-well plates (Corning); and each well contained 1.5×10⁴ peptide-pulsed DCs, 3×10⁵ CD8⁺ T cells and 10 ng/ml of IL-7 (R&D System) in 0.5 ml of AIM-V/2% AS. Three days later, these cultures were supplemented with IL-2 (CHIRON) to a final concentration of 20 IU/ml. On days 7 and 14, the T cells were re-stimulated with the autologous peptide-pulsed DCs. The DCs were prepared by the same way described above. CTLs were tested against peptide-pulsed A24LCL cells or T2 cells after the 3rd round of peptide stimulation on day 21.

CTL Expansion Procedure

CTLs were expanded in culture using a method similar to the one described by Riddell, et al. (Riddel S R, et al. Nature Med. 2: 216-223, 1996; Walter E A, et al. N Engl J. Med. 333: 1038-1044, 1995). A total of 5×10⁴ CTLs were resuspended in 25 ml of AIM-V/5% AS with 2 types of human B-lymphoblastoid cell lines, inactivated by MMC, in the presence of 40 ng/ml of an anti-CD3 monoclonal antibody (Pharmingen). One day after initiating the culture, 120 IU/ml of IL-2 was added to the culture. The culture was fed with fresh AIM-V/5% AS containing 30 IU/ml of IL-2 on days 5, 8 and 11.

Establishment of CTL Clones

The dilutions were made to have 0.3, 1, and 3 CTLs/well in 96 round-bottomed micro titer plate (Nalge Nunc International). CTLs were cultured with 1×10⁴ cells/well of 2 kinds of human B-lymphoblastoid cell lines, 30 ng/ml of anti-CD3 antibody, and 125 U/ml of IL-2 in a total of 150 microliter/well of AIM-V Medium containing 5% AS. 50 microliter/well of IL-2 were added to the medium 10 days later so to reach a final concentration of 125 U/ml IL-2. CTL activity was tested on the 14th day, and CTL clones were expanded using the same method as described above.

Specific CTL Activity

To examine the specific CTL activity, IFN-gamma ELISPOT assay and IFN-gamma ELISA were performed.

Briefly, peptide-pulsed A24-LCL or T2 cell (1×10⁴/well) was prepared as a stimulator cell. Cultured Cells in 48 wells or CTL clones after limiting dilution were used as a responder cells. IFN-gamma ELISPOT assay and ELISA were performed under manufacture procedure.

Results

Prediction of HLA-A24 and HLA-A2 Binding Peptides Derived from STAT3

Table 1 showed the HLA-A*2402 binding peptides for STAT3 in the order of binding affinity from high to low. Table 2 showed the HLA-A*0201 binding peptides for STAT3 in the order of binding affinity from high to low. A total of 32 peptides with potential HLA-A24 binding ability and 71 peptides with potential HLA-A2 binding ability were selected.

TABLE 1
HLA-A2402 binding peptides derived
from STAT3

| | | | | SEQ |
|---|---|---|---|---|
| HLA class | start | | | ID |
| and length | position | sequence | score | NO. |
| STAT3-A24- | 13 | RYLEQLHQL | 720 | 3 |
| 9mer | 93 | RYLEKPMEI | 198 | 4 |
| | 354 | KFPELNYQL | 86.4 | 5 |
| | 78 | LYQHNLRRI | 75 | 6 |
| | 70 | RFLQESNVL | 72 | 7 |
| | 308 | RIVELFRNL | 20.736 | 8 |
| | 344 | QFTTKVRLL | 20 | 9 |
| | 171 | DFDFNYKTL | 20 | 10 |
| | 140 | KQQMLEQHL | 17.28 | 11 |
| | 199 | KMQQLEQML | 17.28 | 12 |
| | 658 | KIMDATNIL | 17.28 | 13 |
| | 350 | RLLVKFPEL | 15.84 | 14 |
| | 25 | SFPMELRQF | 15 | 15 |
| | 180 | KSQGDMQDL | 14.4 | 16 |
| | 262 | RLENWITSL | 12 | 17 |
| | 107 | RCLWEESRL | 12 | 18 |
| | 379 | RGSRKFNIL | 11.52 | 19 |
| | 26 | FPMELRQFL | 10.368 | 20 |
| STAT3-A24- | 21 | LYSDSFPMEL | 264 | 21 |
| 10mer | 445 | VYHQGLKIDL | 240 | 22 |
| | 359 | NYQLKIKVCI | 105 | 23 |
| | 656 | GYKIMDATNI | 50 | 24 |
| | 25 | SFPMELRQFL | 43.2 | 25 |
| | 13 | RYLEQLHQLY | 25.92 | 26 |
| | 511 | QFSSTTKRGL | 20 | 27 |
| | 93 | RYLEKPMEIA | 18 | 28 |
| | 278 | RQQIKKLEEL | 13.2 | 29 |
| | 215 | RSIVSELAGL | 12 | 30 |
| | 107 | RCLWEESRLL | 12 | 31 |
| | 81 | HNLRRIKQFL | 10.08 | 32 |
| | 226 | SAMEYVQKTL | 10.08 | 33 |
| | 312 | LFRNLMKSAF | 10 | 34 |

Start position indicates the number of amino acids from the N terminus of STAT3.

Binding score is derived from “BIMAS” described in Materials and Methods.

TABLE 2
HLA-A0201 binding peptides derived
from STAT3

| | | | | SEQ |
|---|---|---|---|---|
| HLA class | start | | | ID |
| and length | position | sequence | score | NO |
| STAT3-A02- | 223 | GLLSAMEYV | 1595.137 | 35 |
| 9mer | 315 | NLMKSAFVV | 611.96 | 36 |
| | 665 | ILVSPLVYL | 459.398 | 37 |
| | 350 | RLLVKFPEL | 150.178 | 38 |
| | 108 | CLWEESRLL | 145.392 | 39 |
| | 564 | WLDNIIDLV | 144.229 | 40 |
| | 482 | MLTNNPKNV | 118.238 | 41 |
| | 20 | QLYSDSFPM | 92.206 | 42 |
| | 499 | GTWDQVAEV | 75.608 | 43 |
| | 557 | KGFSFWVWL | 64.162 | 44 |
| | 658 | KIMDATNIL | 63.943 | 45 |
| | 283 | KLEELQQKV | 63.877 | 46 |
| | 199 | KMQQLEQML | 53.999 | 47 |
| | 26 | FPMELRQFL | 53.521 | 48 |
| | 2 | AQWNQLQQL | 41.347 | 49 |
| | 403 | SLSAEFKHL | 40.589 | 50 |
| | 385 | NILGTNTKV | 35.385 | 51 |
| | 659 | IMDATNILV | 34.158 | 52 |
| | 87 | KQFLQSRYL | 30.853 | 53 |
| | 269 | SLAESQLQT | 30.553 | 54 |
| | 507 | VLSWQFSST | 24.07 | 55 |
| | 643 | QQLNNMSFA | 23.576 | 56 |
| | 259 | CLDRLENWI | 22.952 | 57 |
| | 304 | MLEERIVEL | 21.917 | 58 |
| | 705 | VLKTKFICV | 21.276 | 59 |
| | 114 | RLLQTAATA | 18.382 | 60 |
| | 360 | YQLKIKVCI | 18.003 | 61 |
| | 201 | QQLEQMLTA | 17.575 | 62 |
| | 143 | MLEQHLQDV | 17.405 | 63 |
| | 578 | ALWNEGYIM | 16.906 | 64 |
| | 205 | QMLTALDQM | 14.962 | 65 |
| | 431 | IVTEELHLI | 14.634 | 66 |
| | 654 | IMGYKIMDA | 14.029 | 67 |
| | 343 | VQFTTKVRL | 13.624 | 68 |
| | 136 | VVTEKQQML | 13.028 | 69 |
| | 469 | QMPNAWASI | 12.809 | 70 |
| | 597 | ILSTKPPGT | 12.668 | 71 |
| | 524 | QLTTLAEKL | 10.468 | 72 |
| STAT3-A02- | 142 | QMLEQHLQDV | 1752.645 | 73 |
| 10mer | 658 | KIMDATNILV | 507.769 | 74 |
| | 554 | MAGKGFSFWV | 199.067 | 75 |
| | 481 | NMLTNNPKNV | 185.858 | 76 |
| | 562 | WVWLDNIIDL | 164.143 | 77 |
| | 664 | NILVSPLVYL | 137.482 | 78 |
| | 201 | QQLEQMLTAL | 75.571 | 79 |
| | 615 | KEGGVTFTWV | 49.464 | 80 |
| | 519 | GLSIEQLTTL | 49.134 | 81 |
| | 507 | VLSWQFSSTT | 48.14 | 82 |
| | 750 | GQFESLTFDM | 44.319 | 83 |
| | 567 | NIIDLVKKYI | 32.348 | 84 |
| | 403 | SLSAEFKHLT | 28.318 | 85 |
| | 77 | VLYQHNLRRI | 26.107 | 86 |
| | 458 | SLPVVVISNI | 23.995 | 87 |
| | 6 | QLQQLDTRYL | 23.499 | 88 |
| | 642 | KQQLNNMSFA | 22.301 | 89 |
| | 524 | QLTTLAEKLL | 21.362 | 90 |
| | 429 | SLIVTEELHL | 21.362 | 91 |
| | 644 | QLNNMSFAEI | 19.822 | 92 |
| | 199 | KMQQLEQMLT | 18.837 | 93 |
| | 114 | RLLQTAATAA | 18.382 | 94 |
| | 337 | LVIKTGVQFT | 14.022 | 95 |
| | 266 | WITSLAESQL | 13.512 | 96 |
| | 26 | FPMELRQFLA | 13.126 | 97 |
| | 340 | KTGVQFTTKV | 12.848 | 98 |
| | 576 | ILALWNEGYI | 12.681 | 99 |
| | 531 | KLLGPGVNYS | 12.208 | 100 |
| | 303 | PMLEERIVEL | 11.843 | 101 |
| | 209 | ALDQMRRSIV | 11.407 | 102 |
| | 308 | RIVELFRNLM | 10.643 | 103 |
| | 222 | AGLLSAMEYV | 10.413 | 104 |
| | 654 | IMGYKIMDAT | 10.311 | 105 |

Start position indicates the number of amino acids from the N terminus of STAT3.

Binding score is derived from “BIMAS” described in Materials and Methods.

Stimulation of the T Cells Using the Predicted Peptides Restricted with HLA-A*2402 or HLA-A*0201

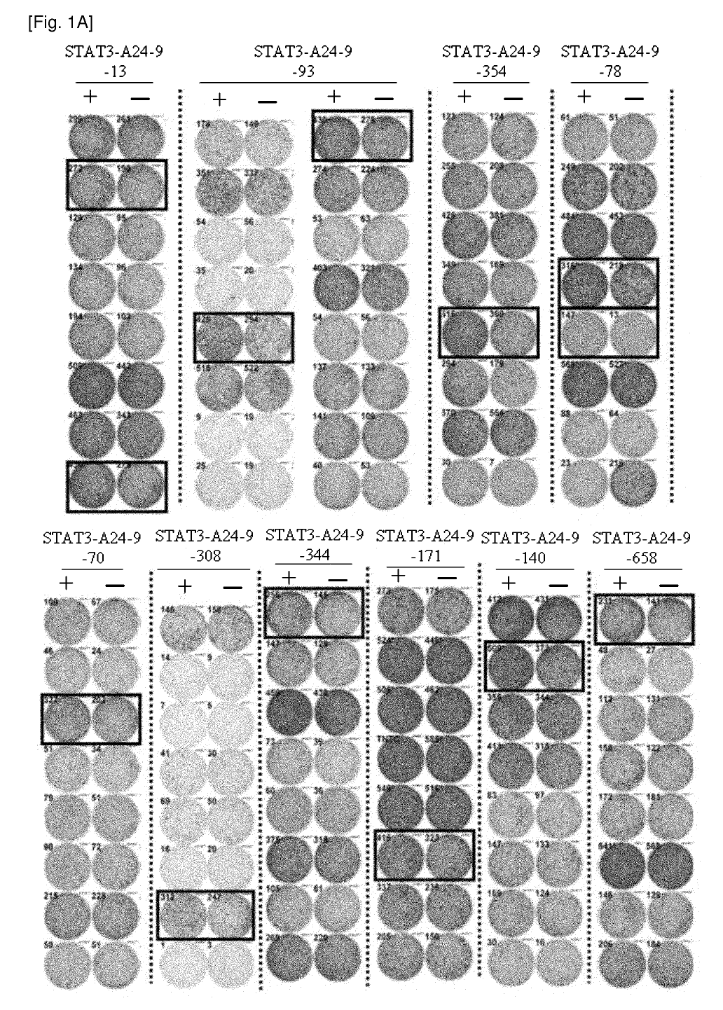

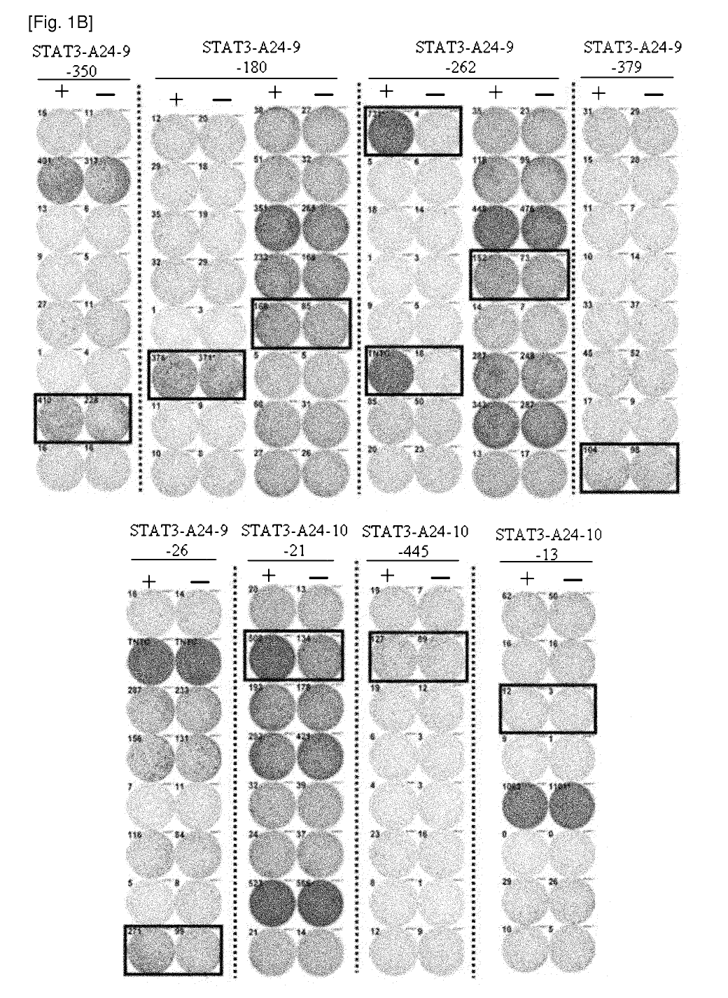

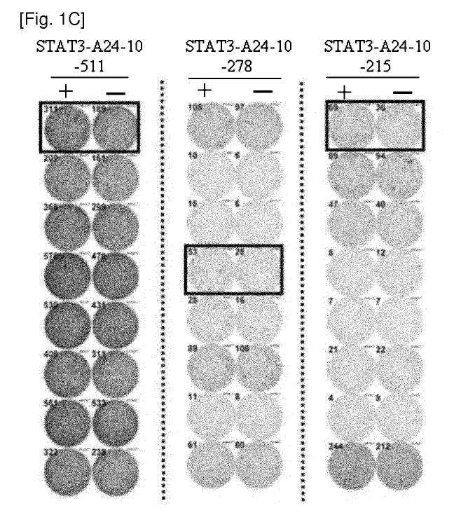

CTLs for those STAT3-derived peptides were generated as described in “Materials and Methods”. As shown in FIGS. 1A to C and 2A to B, the resulting CTLs demonstrated a specific detectable CTL activity by IFN-gamma ELISPOT assay. In FIGS. 1A to C, the cells in the well number No. 2 and No. 8 stimulated with STAT3-A24-9-13 (SEQ ID NO: 3), No. 5 and No. 9 with STAT3-A24-9-93 (SEQ ID NO: 4), No. 5 with STAT3-A24-9-354 (SEQ ID NO: 5), No. 4 and No. 5 with STAT3-A24-9-78 (SEQ ID NO: 6), No. 3 with STAT3-A24-9-70 (SEQ ID NO: 7), No. 7 with STAT3-A24-9-308 (SEQ ID NO: 8), No. 1 with STAT3-A24-9-344 (SEQ ID NO: 9), No. 6 with STAT3-A24-9-171 (SEQ ID NO: 10), No. 2 with STAT3-A24-9-140 (SEQ ID NO: 11), No. 1 with STAT3-A24-9-658 (SEQ ID NO: 13), No. 7 with STAT3-A24-9-350 (SEQ ID NO: 14), No. 6 and No. 13 with STAT3-A24-9-180 (SEQ ID NO: 16), No. 1, No. 6 and No. 12 with STAT3-A24-9-262 (SEQ ID NO: 17), No. 8 with STAT3-A24-9-379 (SEQ ID NO: 19), No. 8 with STAT3-A24-9-26 (SEQ ID NO: 20), No. 2 with STAT3-A24-10-21 (SEQ ID NO: 21), No. 2 with STAT3-A24-10-445 (SEQ ID NO: 22), No. 3 with STAT3-A24-10-13 (SEQ ID NO: 26), No. 1 with STAT3-A24-10-511 (SEQ ID NO: 27), No. 4 with STAT3-A24-10-278 (SEQ ID NO: 29) and No. 1 with STAT3-A24-10-215 (SEQ ID NO: 30) showed potent IFN-gamma production compared with the control. These wells are indicated by boxes in the figures.

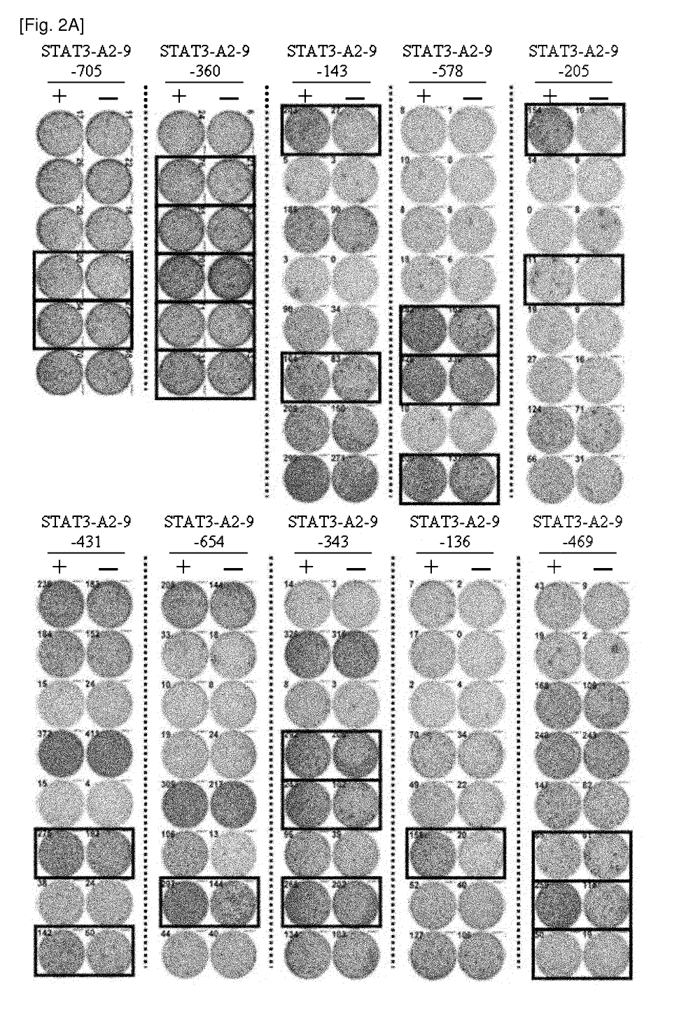

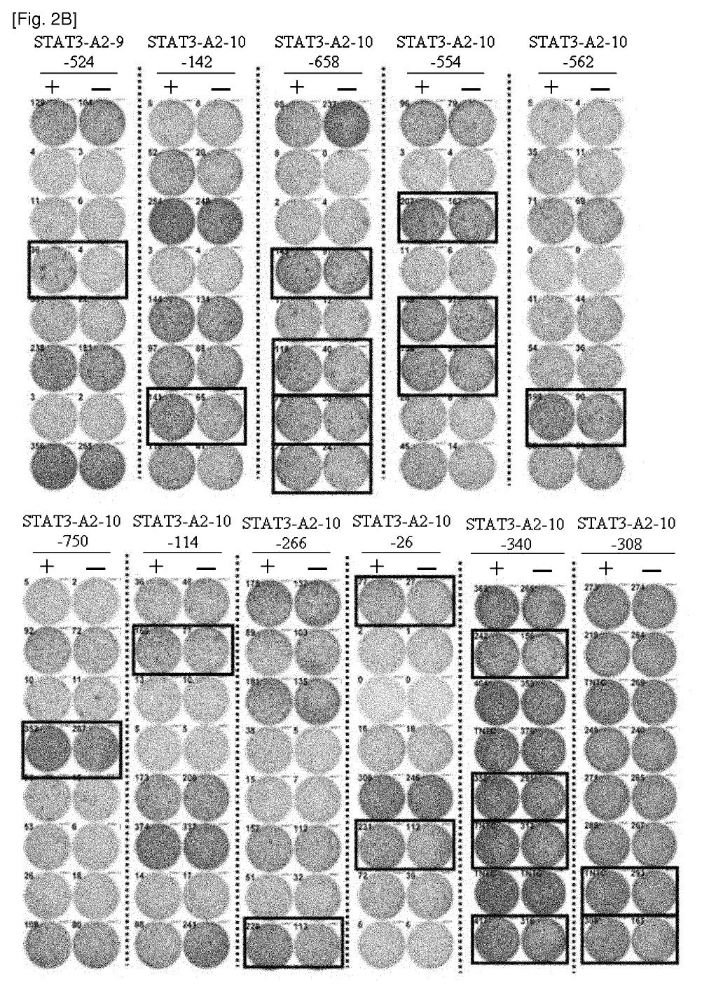

In FIGS. 2A to B, the cells in the well number No. 4 and No. 5 stimulated with STAT3-A2-9-705 (SEQ ID NO: 59), No. 2, No. 3, No. 4, No. 5 and No. 6 with STAT3-A2-9-360 (SEQ ID NO: 61), No. 1 and No. 6 with STAT3-A2-9-143 (SEQ ID NO: 63), No. 5, No. 6 and No. 8 with STAT3-A2-9-578 (SEQ ID NO: 64), No. 1 and No. 4 with STAT3-A2-9-205 (SEQ ID NO: 65), No. 6 and No. 8 with STAT3-A2-9-431 (SEQ ID NO: 66), No. 7 with STAT3-A2-9-654 (SEQ ID NO: 67), No. 4, No. 5 and No. 7 with STAT3-A2-9-343 (SEQ ID NO: 68), No. 6 with STAT3-A2-9-136 (SEQ ID NO: 69), No. 6, No. 7 and No. 8 with STAT3-A2-9-469 (SEQ ID NO: 70), No. 4 with STAT3-A2-9-524 (SEQ ID NO: 72), No. 7 with STAT3-A2-10-142 (SEQ ID NO: 73), No. 4, No. 6, No. 7 and No. 8 with STAT3-A2-10-658 (SEQ ID NO: 74), No. 3, No. 5 and No. 6 with STAT3-A2-10-554 (SEQ ID NO: 75), No. 7 with STAT3-A2-10-562 (SEQ ID NO: 77), No. 4 with STAT3-A2-10-750 (SEQ ID NO: 83), No. 2 with STAT3-A2-10-114 (SEQ ID NO: 94), No. 8 with STAT3-A2-10-266 (SEQ ID NO: 96), No. 1 and No. 6 with STAT3-A2-10-26 (SEQ ID NO: 97), No. 2, No. 5, No. 6 and No. 8 with STAT3-A2-10-340 (SEQ ID NO: 98) and No. 7 and No. 8 with STAT3-A2-10-308 (SEQ ID NO: 103) showed potent IFN-gamma production compared with the control. These wells are indicated by boxes in the figures.

Establishment of CTL Lines Stimulated with Peptides Derived from STAT3

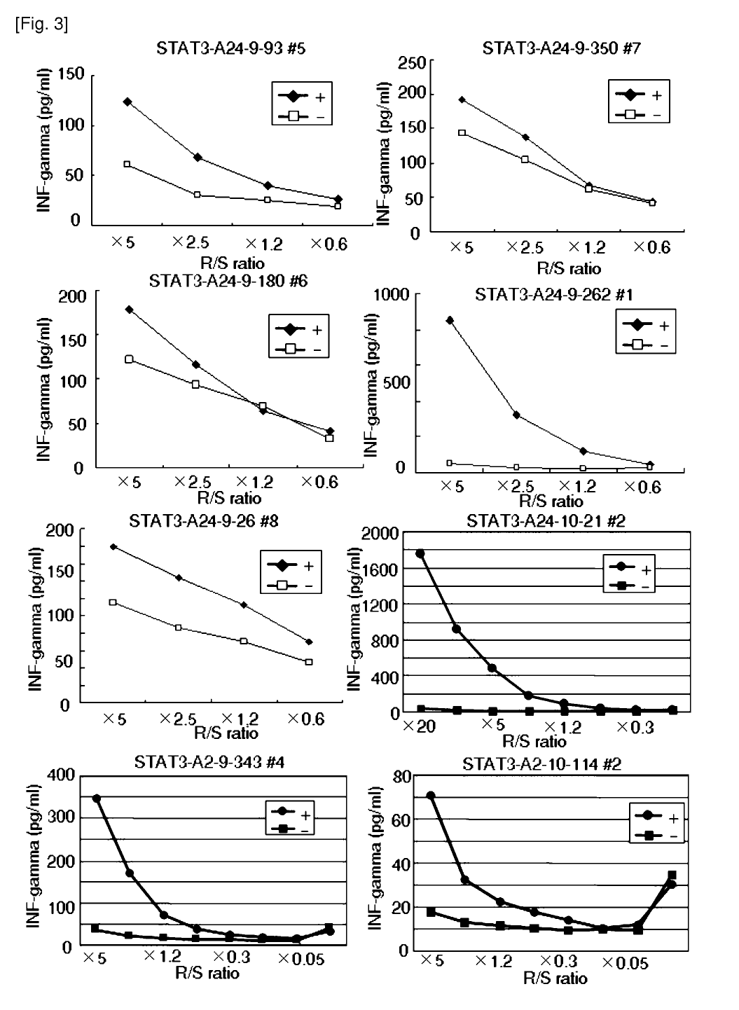

From the positive wells in the 1st screening, we established CTL lines stimulated with the HLA-A24 restricted peptides derived from STAT3. In FIG. 3, the ratio IFN-gamma production derived from CTL lines stimulated with STAT3-A24-9-93 (SEQ ID NO: 4), STAT3-A24-9-350 (SEQ ID NO: 14), STAT3-A24-9-180 (SEQ ID NO: 16), STAT3-A24-9-262 (SEQ ID NO: 17), STAT3-A24-9-26 (SEQ ID NO: 20) and STAT3-A24-10-21 (SEQ ID NO: 21) peptide was detected by IFN-gamma ELISA assay. These data show that STAT3-A24-9-93 (SEQ ID NO: 4), STAT3-A24-9-350 (SEQ ID NO: 14), STAT3-A24-9-180 (SEQ ID NO: 16), STAT3-A24-9-262 (SEQ ID NO: 17), STAT3-A24-9-26 (SEQ ID NO: 20) and STAT3-A24-10-21 (SEQ ID NO: 21) peptide was able to induce a specific CTL response. Correspondingly, the IFN-gamma production derived from CTL lines stimulated with the HLA-A2 restricted peptides, STAT3-A2-9-343 (SEQ ID NO: 68) and STAT3-A2-10-114 (SEQ ID NO: 94), were detected by IFN-gamma ELISA assay. These data show that STAT3-A2-9-343 (SEQ ID NO: 68) and STAT3-A2-10-114 (SEQ ID NO: 94) peptides were able to induce specific CTL responses.

Establishment of CTL Clones Stimulated with Peptides Derived from STAT3

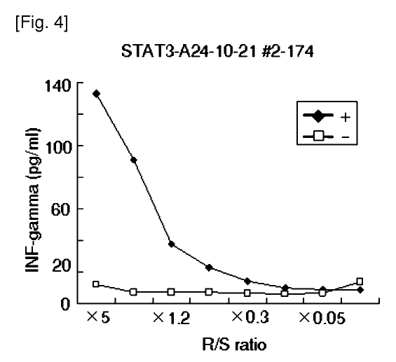

CTL clones were established by limiting dilution from CTL lines as described in “Materials and Methods”, and ratio dependent IFN-gamma production from CTL clones against target cells pulsed peptide were determined by IFN-gamma ELISA assay. Potent IFN-gamma productions were determined from CTL clones stimulated with STAT3-A24-10-21 (SEQ ID NO: 21) in FIG. 4.

Homology Analysis of the Antigen Peptides

The CTLs stimulated with the following peptides showed significant and specific CTL activities.

STAT3-A24-9-13 (SEQ ID NO: 3),

STAT3-A24-9-93 (SEQ ID NO: 4),

STAT3-A24-9-354 (SEQ ID NO: 5),

STAT3-A24-9-78 (SEQ ID NO: 6),

STAT3-A24-9-70 (SEQ ID NO: 7),

STAT3-A24-9-308 (SEQ ID NO: 8),

STAT3-A24-9-344 (SEQ ID NO: 9),

STAT3-A24-9-171 (SEQ ID NO: 10),

STAT3-A24-9-140 (SEQ ID NO: 11),

STAT3-A24-9-658 (SEQ ID NO: 13),

STAT3-A24-9-350 (SEQ ID NO: 14),

STAT3-A24-9-180 (SEQ ID NO: 16),

STAT3-A24-9-262 (SEQ ID NO: 17),

STAT3-A24-9-379 (SEQ ID NO: 19),

STAT3-A24-9-26 (SEQ ID NO: 20),

STAT3-A24-10-21 (SEQ ID NO: 21),

STAT3-A24-10-445 (SEQ ID NO: 22),

STAT3-A24-10-13 (SEQ ID NO: 26),

STAT3-A24-10-511 (SEQ ID NO: 27),

STAT3-A24-10-278 (SEQ ID NO: 29),

STAT3-A24-10-215 (SEQ ID NO: 30),

STAT3-A2-9-705 (SEQ ID NO: 59),

STAT3-A2-9-360 (SEQ ID NO: 61),

STAT3-A2-9-143 (SEQ ID NO: 63),

STAT3-A2-9-578 (SEQ ID NO: 64),

STAT3-A2-9-205 (SEQ ID NO: 65),

STAT3-A2-9-431 (SEQ ID NO: 66),

STAT3-A2-9-654 (SEQ ID NO: 67),

STAT3-A2-9-343 (SEQ ID NO: 68),

STAT3-A2-9-136 (SEQ ID NO: 69),

STAT3-A2-9-469 (SEQ ID NO: 70),

STAT3-A2-9-524 (SEQ ID NO: 72),

STAT3-A2-10-142 (SEQ ID NO: 73),

STAT3-A2-10-658 (SEQ ID NO: 74),

STAT3-A2-10-554 (SEQ ID NO: 75),

STAT3-A2-10-562 (SEQ ID NO: 77),

STAT3-A2-10-750 (SEQ ID NO: 83),

STAT3-A2-10-114 (SEQ ID NO: 94),

STAT3-A2-10-266 (SEQ ID NO: 96),

STAT3-A2-10-26 (SEQ ID NO: 97),

STAT3-A2-10-340 (SEQ ID NO: 98) and

STAT3-A2-10-308 (SEQ ID NO: 103).

To exclude unexpected reaction for other proteins that have homologous sequence to these epitope peptides, homology analysis was performed with the peptide sequences as queries using the BLAST algorithm (http://www.ncbi.nlm.nih.gov/blast/blast.cgi) and revealed no significant sequence homology with known proteins.

These results indicate that the CTLs stimulated with these epitope peptides have high specificity and are unlikely to raise unintended immunologic response to unrelated molecules.

### DISCUSSION

Identification of new TAAs, which induce potent and specific anti-tumor immune responses, guarantees further development of clinical applications of the peptide vaccination strategy in various types of cancer (Harris C C. J Natl Cancer Inst 88:1442-1445, 1996; Butterfield L H, et al. Cancer Res 59:3134-3142, 1999; Vissers J L M, et al, Cancer Res 59: 5554-5559, 1999; Van den Burg S H, et al. J. Immunol. 156:3308-3314, 1996; Tanaka F, et al. Cancer Res 57:4465-4468, 1997; Fujie T, et al. Int J Cancer 80:169-172, 1999; Kikuchi M, et al. Int J Cancer 81: 459-466, 1999; Oiso M, et al. Int J Cancer 81:387-394, 1999). Various types of antigen-specific immunotherapy have been performed; however, clinical efficacy is not always as high as expected (Rosenberg S A, et al. Nature Med. 10:909-915, 2004).

To improve the clinical efficacy for immunotherapy, it is important to overcome the immunosuppressive factors induced by tumor as well as to elicit CTLs against tumor. It has been reported that tumor-infiltrating immature myeloid cells and immature dendritic cells, immunosuppressive cells against the anti-tumor immune system, have high expression of STAT3 (Yu H, et al. Nat Rev Immunol. 7: 41-51, 2007; Kortylewski M, et al. Nature Med. 11:1314-1321, 2005; Jing N and Tweardy D J. Anti-Cancer Drugs. 16: 601-607, 2005). Recent studies have shown that blocking STAT3 signaling decreases the number of immature DCs and accelerates functional maturation of DCs (Wang T, et al. Nature Med. 10: 48-54, 2004).

To overcome this problem, the peptides derived from STAT3 as antigen epitopes were analyzed as to whether they are restricted by HLA-A24 and HLA-A2, which are common HLA alleles in the human population (Date Y, et al. Tissue Antigens, 47: 93-101, 1996; Kondo A, et al. J Immunol, 155: 4307-4312, 1995; Kubo R T, et al. J Immunol, 152: 3913-3924, 1994). In this invention, the candidates of HLA-A24- and HLA-A2-binding peptides derived from STAT3 were predicted using the information of their binding affinities to HLA-A*2402 and HLA-A*0201. After the in vitro stimulation of T-cells by DCs loaded with these peptides, CTLs were successfully established using the following peptides:

STAT3-A24-9-13 (SEQ ID NO: 3),

STAT3-A24-9-93 (SEQ ID NO: 4),

STAT3-A24-9-354 (SEQ ID NO: 5),

STAT3-A24-9-78 (SEQ ID NO: 6),

STAT3-A24-9-70 (SEQ ID NO: 7),

STAT3-A24-9-308 (SEQ ID NO: 8),

STAT3-A24-9-344 (SEQ ID NO: 9),

STAT3-A24-9-171 (SEQ ID NO: 10),

STAT3-A24-9-140 (SEQ ID NO: 11),

STAT3-A24-9-658 (SEQ ID NO: 13),

STAT3-A24-9-350 (SEQ ID NO: 14),

STAT3-A24-9-180 (SEQ ID NO: 16),

STAT3-A24-9-262 (SEQ ID NO: 17),

STAT3-A24-9-379 (SEQ ID NO: 19),

STAT3-A24-9-26 (SEQ ID NO: 20),

STAT3-A24-10-21 (SEQ ID NO: 21),

STAT3-A24-10-445 (SEQ ID NO: 22),

STAT3-A24-10-13 (SEQ ID NO: 26),

STAT3-A24-10-511 (SEQ ID NO: 27),

STAT3-A24-10-278 (SEQ ID NO: 29),

STAT3-A24-10-215 (SEQ ID NO: 30),

STAT3-A2-9-705 (SEQ ID NO: 59),

STAT3-A2-9-360 (SEQ ID NO: 61),

STAT3-A2-9-143 (SEQ ID NO: 63),

STAT3-A2-9-578 (SEQ ID NO: 64),

STAT3-A2-9-205 (SEQ ID NO: 65),

STAT3-A2-9-431 (SEQ ID NO: 66),

STAT3-A2-9-654 (SEQ ID NO: 67),

STAT3-A2-9-343 (SEQ ID NO: 68),

STAT3-A2-9-136 (SEQ ID NO: 69),

STAT3-A2-9-469 (SEQ ID NO: 70),

STAT3-A2-9-524 (SEQ ID NO: 72),

STAT3-A2-10-142 (SEQ ID NO: 73),

STAT3-A2-10-658 (SEQ ID NO: 74),

STAT3-A2-10-554 (SEQ ID NO: 75),

STAT3-A2-10-562 (SEQ ID NO: 77),

STAT3-A2-10-750 (SEQ ID NO: 83),

STAT3-A2-10-114 (SEQ ID NO: 94),

STAT3-A2-10-266 (SEQ ID NO: 96),

STAT3-A2-10-26 (SEQ ID NO: 97),

STAT3-A2-10-340 (SEQ ID NO: 98) and

STAT3-A2-10-308 (SEQ ID NO: 103).

The resulting CTLs showed a specific potent CTL activity against the peptide-pulsed target cells. These results demonstrate that STAT3 is a novel antigen recognized by CTLs and that these epitope peptides of STAT3 are restricted by HLA-A24 and HLA-A2. Since STAT3 is expressed in tumor and associated with immunosuppression, STAT3 is therefore a good target for immunotherapy to promote clinical efficacy.

Homology analysis of the STAT3 epitope peptides, which can induce CTLs, showed that they do not have significant homology with peptides derived from any other known human gene products. In conclusion, the STAT3 epitope peptides discovered in this invention are useful as cancer vaccine for cancer immunotherapy.

### INDUSTRIAL APPLICABILITY

To improve the clinical efficacy for immunotherapy, it is important to overcome the immunosuppressive factors induced by the tumor. T-regs are thought to be one of the major players to suppress the various types of immune responses. To inhibit tumor growth, it is also important to suppress angiogenesis around the tumor lesion. Angiogenesis-associated factors are produced by tumor cells or inflammatory cells. For example, the present invention provides a novel therapeutic strategy for treating solid cancers which require angiogenesis for their progression. Alternatively, cancers which depend on immune tolerance for their survival may also be treated by the present invention. Therefore, it is crucial to develop vaccines that target STATS-expressing cells to overcome immunosuppression and angiogenesis.

1. (canceled)

2. A peptide, which is selected from the group consisting of:
(a) a nonapeptide or decapeptide selected from peptides comprising the amino acid sequence of SEQ ID NO: 3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98 or 103; and
(b) a peptide having cytotoxic T cell inducibility, wherein the peptide comprises one, two, or several amino acid substitutions or additions in the amino acid sequence of SEQ ID NO:3, 4, 5, 6, 7, 8, 9, 10, 11, 13, 14, 16, 17, 19, 20, 21, 22, 26, 27, 29, 30, 59, 61, 63, 64, 65, 66, 67, 68, 69, 70, 72, 73, 74, 75, 77, 83, 94, 96, 97, 98, or 103.

3. (canceled)

4. The peptide of claim 2, wherein the second amino acid from the N terminus of SEQ ID NO: 3, 4, 5, 6, 7, 8, 9, 10, 11, 13, 14, 16, 17, 19, 20, 21, 22, 26, 27, 29 or 30, is substituted with phenylalanine, tyrosine, methionine, or tryptophan.

5. The peptide of claim 2, wherein the C-terminal amino acid of SEQ ID NO: 3, 4, 5, 6, 7, 8, 9, 10, 11, 13, 14, 16, 17, 19, 20, 21, 22, 26, 27, 29 or 30, is substituted with phenylalanine, leucine, isoleucine, tryptophan or methionine.

6. The peptide of claim 2, wherein the second amino acid from the N terminus of SEQ ID NO: 59, 61, 63, 64, 65, 66, 67, 68, 69, 70, 72, 73, 74, 75, 77, 83, 94, 96, 97, 98 or 103, is substituted with leucine or methionine.

7. The peptide of claim 2, wherein the C-terminal amino acid of SEQ ID NO: 59, 61, 63, 64, 65, 66, 67, 68, 69, 70, 72, 73, 74, 75, 77, 83, 94, 96, 97, 98 or 103, is substituted with valine or leucine.

8. An agent for inducing cytotoxic T cells, wherein the agent comprises one or more peptides of claim 2.

9. A pharmaceutical composition for treating or preventing cancer, wherein the composition comprises one or more peptides of claim 2.

10. An exosome that presents on its surface a complex comprising the peptide of claim 2 and an HLA antigen.

11. A method of preparing antigen-presenting cells having cytotoxic T cell inducibility, the method comprising contacting one or more peptides of claim 2 with antigen-presenting cells, or transferring a gene(s) comprising a polynucleotide encoding the peptide into antigen-presenting cells.

12. A method of preparing cytotoxic T cells, the method comprising contacting T cells with antigen-presenting cells presenting one or more peptides of claim 2.

13. A method of treating or preventing cancer by administering a composition comprising one or more peptides of claim 2.

14. An isolated cytotoxic T cell prepared by the method of claim 12.

15. An antigen-presenting cell comprising a complex formed between an HLA antigen and the peptide of claim 2.

16. An antigen-presenting cell prepared by the method of claim 11.

17. A vaccine for inhibiting angiogenesis or regulating regulatory T cells, wherein the vaccine comprises at least one peptide of claim 2 as an active ingredient.

18. A method of inhibiting angiogenesis or regulating regulatory T cells by administering to a subject, a vaccine comprising at least one peptide of claim 2.

19. A vaccine for enhancing clinical efficacy of cancer immunotherapy, wherein the vaccine comprises at least one peptide of claim 2 as an active ingredient.

20. A method of enhancing clinical efficacy of cancer immunotherapy by administering to a subject, a vaccine comprising one or more peptides of claim 2, or an immunologically active fragment of said peptide, or a polynucleotide encoding said peptide.

21. An isolated cytotoxic T cell transduced with a nucleic acid encoding a polypeptide of a TCR subunit that binds with the peptide of claim 2 in the context of HLA-A24 or HLA-A2.

22. A composition comprising one or more peptides of claim 2.
